(19) United States
(12) Patent Application Publication
(10) Pub. No.: US 2018/0284108 A1
(43) Pub. Date: Oct. 4, 2018

(54) METHOD FOR COMPLETE AND FRAGMENTED MARKERS

(71) Applicants: Michael Joseph Pugia, Ganger, IN (US); Zane Baird, Brigham City, UT (US); Zehui Cao, Carmel, IN (US)

(72) Inventors: Michael Joseph Pugia, Ganger, IN (US); Zane Baird, Brigham City, UT (US); Zehui Cao, Carmel, IN (US)

(21) Appl. No.: 15/942,524

(22) Filed: Mar. 31, 2018

Related U.S. Application Data
(60) Provisional application No. 62/480,370, filed on Apr. 1, 2017.

(51) Int. Cl. G01N 33/543

(52) U.S. Cl. CPC G01N 33/54306; G01N 33/54353; G01N 33/54313

(57) ABSTRACT

The invention described herein is directed to methods of isolation of all variations of analyte in a sample by binding variations to a particle with attached analytical labels and separating the particles from the sample followed by removing analytical labels from particle and measuring the analyte molecules by the measuring the analytical labels. The separated analytical labels on the particle are then able to be used to measure the variations of analyte binding variations.

This application claims the priority benefit under 35 U.S.C. section 119 of U.S. provisional patent application No. 62/480,370 entitled “Method For Complete And Fragmented Markers” filed on Apr. 1, 2017; and which is in its entirety herein incorporated by reference.

### BACKGROUND

The invention relates to methods for enriching and detecting rare molecules relative to non-rare molecules. In some aspects the invention relates to methods, apparatus and kits for detecting one or more different populations of rare molecules in a sample suspected of containing one or more different populations of rare molecules and non-rare molecules. In some aspects, the invention relates to methods and kits for detecting one or more different populations of rare molecules that are freely circulating in samples. In other aspects, the invention relates to methods and kits for detecting one or more different populations of rare molecules that are associated with rare cells in a sample suspected of containing the one or more different populations of rare cells and non-rare cells.

The detection of rare molecules in the range of 1 to 50,000 copies per 10 μL (femtomolar (fM) or less) cannot be achieved by conventional affinity assays, which require molecular copy numbers far above those found for rare molecules. For example, immunoassays cannot typically achieve a detection limit of 1 picomolar (pM) or less. Immunoassays are limited by the affinity binding constant of an antibody, which is typically not higher than 10⁻¹² (1 pM). Immunoassays require at least 100-fold antibody excess as the off-rate is generally 10⁻¹³ and a complete binding of all analyte in a sample is limited by antibody solubility. This same issue of antibody solubility prevents conventional immunoassays from reaching sub-attomolar detection levels.

The detection of rare molecules that are cell-bound or contained within a cell is also important in medical applications such as in the diagnosis of diseases that can be propagated from a single cell. The detection of circulating rare molecules is complicated by the sample containing a mixture of rare and non-rare molecules. The materials can be cellular, e.g. internal to cells or “cell free” material and not bound or associated to any intact cell. Cell free rare molecules are important in medical applications such as, for example, diagnosis of cancer in tissues. In the case of cancer, rare molecules are shed from tissues into circulation and it is understood that cell free rare molecules correlates to the total amount of rare molecules in diseased tissues, for example tumor, distributed throughout the body. Cell free analysis requires isolation and detection of circulating rare molecules from a very small fraction of all molecules in a sample. When cell free molecules are shed into the peripheral blood from diseased cells in tissues, these molecules are mixed with molecules shed from healthy cells. For example, approximately 109 cells are present in 1 cm³ of diseased tissue. If this tissue mass was fully dissolved into 5 L of blood (blood volume of an average adult) this would only be 2 million cells per 10 mL blood and would be considered rare, considering there are an average of 75 million leukocytes and 50 billion erythrocytes per 10 mL blood, each of which releases non-rare molecules.

The complexity of peptide and protein variations in samples causes significant issues when a measurement of the respective proteins and peptides is desired. These issues of variation have been demonstrated using the SELDI affinity mass spectroscopic method in a study which utilized antibodies for peptide and protein isolation (Pugia, Glycoconj J 2007). Peptides and proteins are known to fragment and to undergo post-translational modifications in biological systems under the action of enzymes. For example, a high degree of variations of urinary trypsin inhibitor was detected in biological samples of different patients as the result of fragmentation and glyco-conjugation with hundreds of different forms detected. The forms detected depended on the patient, disease, sample type, and affinity agent used for isolation. Unique affinity agents exhibited different cross reactivity to other proteins. This variation causes problems for analysis. For example, the measurement of separate, unique fragments originating from the same peptide or protein often produces different results. Determination of which fragments are more or less significant is needed, the summation of similar fragments might be required, and affinity reagents used for methods can be more or less reactive to certain fragments. The variation of peptides and proteins increases as these variants become bound by other biomolecules which can alter the function of the variants.

The high degree of variations in peptides and proteins becomes a problem as immunoassay methods must often be able detect each variant independently. Sandwich immunoassays are typically used for specifically measuring unique fragments or forms of an analyte and rely on measuring a variation by binding two separate locations. Sandwich immunoassays require adequate space for two separate antibodies to bind the same fragment; however, as these fragments contain the same peptide or protein regions as those other variants, regions are often unsuited for binding to antibodies for specific assays. Additional binding by other biomolecules can be blocking to antibodies or cause cross reactivity. Cysteine may form disulfide bonds and other secondary molecules can bind fragments or be cleaved and alter antibody binding, to name a few of the problems in the measurement of peptides and proteins with a high degree of variation by immunoassay. Multiplexing is another problem for immunoassay methods as most methods use optical detection labels—whether chemiluminescent, fluorescent, or colorimetric—which provide a limited number of resolvable signals for simultaneous measurement within the same analysis. For this reason, analysis of hundreds to thousands of variations is a problem for optical systems. These methods require multiple, separate measurements in multiplexed panels and arrays which increases cost and complexity.

Common alternative approaches to solve the problem of high degrees of variations is through the use of the peptide or protein to be measured as a substrate for the action of enzymes, proteases and peptidases. These measurements are based on the observed protease activity and can be used to measure the enzymes, proteases, peptidases and inhibitors thereof. For example, these methods have been used to analyze serine proteases of the trypsin family (Elastase, Cathepsin, Tryptase, Trypsin, Kallikrein, Thrombin, Plasmin and Factors VII & X) and their inhibitors (Bikunin, Uristatin, and Urinary Trypsin Inhibitor) (Corey U.S. Pat. No. 6,955,921). In these cases the peptide is used as a substrate, attached to a chromophore at the amino acid cleavage site. Upon cleavage by the protease, a fragment is released and activated to generate a color. The concentration of inhibitor is measured when a known amount of protease is added. Here the amount of inhibitor is inversely proportional to the amount of substrate released, since the inhibitor decreases the activity of protease. The chromophores however are sensitive to interference where color is reversed or prematurely generated by sample pH, oxidants, reductants, or reactants.

The use of mass spectroscopy to measure the peptide or protein substrate has been used to eliminate the issues associated with chromophores. For example this has been shown for the renin-angiotensin-aldosterone system. In this system angiotensinogen I (Ang I) (DRVYIHPFHL) is converted to Ang II (DRVYIHPF) by the cleavage of two C-terminal amino acids in an enzymatic cleavage by renin (Popp 2014). Measurements of Ang I allows for a plasma renin activity assay by utilizing anti-Ang I antibodies immobilized to affinity beads to simultaneously capture endogenous Ang I from plasma along with a stable isotope-labeled Ang I. The plasma sample is split and incubated either at 37° C. for 3 h or on ice. A determination of the difference in Ang I concentration for the two plasma incubation conditions allows the calculation of the patient's plasma renin activity. This enzyme, protease and peptidase assay is still sensitive to interference where activity are inhibited or activated by sample pH, sample stability, inhibitors, co-factors, time and temperatures

Mass spectrometry (MS) is an extremely sensitive and specific technique very well suited for detecting small molecules) down to pM concentrations with small sample consumption (1 microliter (μL) or less). MS also has the ability to simultaneously measure hundreds of components (multiplexing) present in complex biological media in a single assay without the need for labeled reagents. The method offers specificity and sensitivity until the biological complexity causes overlapping signals (isobaric interference) or results in ion suppression. The coupling of MS with a pre-separation step such as liquid chromatography (LC-MS) is a widely used method of increasing sensitivity and limiting isobaric interference, and overcoming ion suppression by high abundance non-analyte sample components; however this greatly increases analytical run time, cost, and sample preparation complexity. Tandem MS (MS/MS) can be used to both increase signal-to-noise in the case of high background interference as well as distinguish isobaric analytes (share the same parent mass-to-charge (m/z)) but exhibit unique fragmentation within the mass spectrometer; however, analysis of MS/MS data is not a simple task, especially in the case of post-translationally modified proteins and peptides and still suffers the effects of ion suppression, especially in the case of poorly ionizable fragments. Matrix-assisted laser desorption/ionization using a time-of-flight mass spectrometer (MALDI-TOF) is well suited for high sensitivity analysis of low abundance molecules; however, sample complexity and matrix interference frequently results in isobaric interference.

The current state of MS is not competitive with routine clinical diagnostic systems, with noted problems in the inability to separate markers of interest (sample preparation), loss of sensitivity due to high background in clinical samples, inefficient ionization of some fragments, and isobaric interference in complex samples such as blood. In addition, MS is often unable to detect certain masses due to ion suppression by more easily ionizable molecules present in the sample. These issues typically cause false results.

A proteolytic digestion is often utilized for the analysis and quantitation of proteins and peptides by MS. The digestion serves to break the protein or peptide into smaller, more easily detectable fragments that can be better separated before MS analysis as is the case with LC-MS. While serving to increase analytical sensitivity, proteolytic digestion is often not reproducible—not all proteins and bound forms can be fragmented, certain fragments are not easily detected (method is biased towards easily ionizable fragments), various matrix components can inhibit the digestion enzymes used, and redundant amino acid sequences can result in ambiguity during data analysis. Fragments detected under these conditions often do not relate to the clinical state as they are not the relevant molecule regions. Additionally, quantitation of fragments requires the inclusion of a stable isotope internal standard.

One approach to solve the problems of sensitivity and quantitation by MS is to chemically add a label to the molecule to be measured (Demmer 2012). This mass labeling approach has been helpful in the detection of cells, tissues, peptides, and proteins by mass spectrometry. Chemical labeling works by introducing a charged group of known mass directly on the molecule to be measured through a chemical reaction. While these mass labeling approaches allow masses to be more easily ionized and uniquely identified, they still suffer from the effects of isobaric interference, require the analyte to have a functional group amenable to mass label introduction, and are limited by the mass of the analyte to be measured. Therefore, other approaches were sought to avoid or reduce the problems associated with these current mass spectral analysis methods.

One common approach utilizes affinity agents to capture an analyte and remove contaminates prior to detection by MS, often termed affinity mass spectrometry. One method of affinity mass spectrometry is Surface Enhanced Laser Desorption and Ionization or SELDI (U.S. Pat. No. 5,719,060 and U.S. Pat. No. 6,225,047, both to Hutchens and Yip). This method uses affinity agents to specifically absorb analytes to a surface which aids in the ionization of captured molecules (Zhu 2006). Other examples include affinity agents on a solid substrate, either flexible or rigid, that has a sample-presenting surface. Other “affinity mass spectrometry” methods use an affinity agent, like an antibody, attached to a capture surface or particle for isolation into liquids followed by ionization. While these methods have been successfully used for clinical measurement (Popp 2014), they often require enzymatic digestion in order to produce fragments detectable by MS. This method of sample preparation remains a difficult and complex multistep process to automate and is noncompetitive with other detection technologies used in the clinical laboratory.

A mass labeling approach which utilizes affinity agents has been accomplished through the coupling of metals to antibodies against rare cell molecules of interest (Bandura 2009, Lee 2008). In this instance the entire sample is subjected to atomization and the metal content is used to assay the presence of the rare molecule, which results in the destruction of the entire sample. In Pugia PCT/US2015/033278 a quaternary ammonium compound is attached to a nanoparticle through disulfide bonds. The nanoparticle is also conjugated to affinity agents for rare molecules. Here a chemical is used as an “alteration agent” to release the mass label from the affinity agent by breaking a disulfide bond, namely dithiothreitol (DTT) or tris(2-carboxyethyl)phosphine (TCEP). This method allows sensitivities in the μM range to detect a limited number of peptide and protein variants in a sample. Combining affinity agents and mass labeling for mass spectrometry using a nanoparticle and mass label is shown in Cooks PCT/US16/53610 filed Sep. 24, 2016. In this example, an affinity tag and a mass label with a quaternary ammonium group is connected to a particle by a cleavable ketal linkage. This method uses the affinity tag to connect to an affinity agent. While this method allows high sensitivities in nM range to detect limited number of peptide and proteins variants in a sample, it suffered from a lack of specificity due to the affinity tag binding to non-analyte molecules. This made the method unable to accurately measure all the variation of an analyte and therefore result in false positives.

Some labeling strategies such as isobaric tags for relative and absolute quantitation (iTRAQ™, SCIEX) or tandem mass tags (TMT™, Thermo Scientific) offer a direct labeling approach that is amenable to multiplexed sample measurement and relative quantitation. In both TMT and iTRAQ separate proteolytic digests are reacted with reagents which introduce unique charged groups onto N-terminal amino acids, as well as cysteine, lysine, and carbonyl moieties. The labeled samples are then pooled and analyzed in the same LC-MS run. The result is a multiplexable (up to 10 plex) assay capable of relative quantification within the same LC-MS analysis. The reagents enable multiplexing by producing isobaric, chromatographically indistinguishable, derivatized peptides which produce unique reporter ions for identical peptides from different samples analyzed in the same pool. As this method still relies on pre-separation by LC, proteolytic digestion, as well as the added complexity of independent sample derivatization it is subject to the same problems associated with the previously discussed methods.

The field requires an improved method capable of detecting all variations of peptides and proteins in a sample. This method should not be dependent on further enzymatic processing, peptidase reactions, and be able to measure any and all variations of an analyte in a single determination. A new method which combines affinity agents and analytical labeling must be sensitive to variations of peptide and proteins in a sample and allow for consistent measurement across patients and samples.

### SUMMARY OF THE INVENTION

The invention described herein is directed to methods of isolation of variations of analyte molecules in a sample by binding variations to a particle with attached analytical labels and affinity agents, separation of the particles from the sample, removal of analytical labels from particles, and subsequent measurement of the analytical labels for indirect analysis of analyte molecules.

Some examples in accordance with the invention are directed to a method of isolating all variations of analyte in a sample by binding all variation of analyte to particles which host an analytical label; where multiple identical analytical labels are attached to a particle by an X-Y bond and are released by breaking the X-Y bond.

Some examples in accordance with the invention are directed to a method of isolation of first variation of analyte in a sample by binding the first variation of analyte to a particle with a first analytical label; additional variations of analyte are further bound to particles with additional analytical labels where all analytical labels are attached an X-Y bond and released by breaking the X-Y bond.

Some examples in accordance with the invention are directed to a method of isolating variations of analyte in a sample by binding all variation of analyte to particles with analytical label; where multiple identical affinity agents are attached to particles by and X-Y bond but are not released by conditions breaking the X-Y bond.

Some examples in accordance with the invention are directed to methods of isolation of a first variation of analyte in a sample by binding the first variation of analyte to particle with a first affinity agent; additional variations of analyte are further bound to particles with additional affinity agent where all affinity agents are attached an X-Y bond but are released by breaking the X-Y bond.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a schematic illustrating an example of the formation of variations of analyte which is detected by an apparatus, method, or kit in accordance with the principles described herein. The formation of the original form of the analyte, such as the gene product 1 is acted on by a group of agents 2 able to generate a variation of the analyte by fragmentation (such as proteases) which lead to 10 or more fragments 3. The variations of analyte achieved by fragmentation 3 is acted on by a group of agents 4 able to generate variations of the analyte to 10 or more additional variations 5. The variations of analyte by additions 5 is acted on group of agents 6 able to generate a variation of the analyte by binding such as protein to 10 or more additional variations 7 able to generate a variation of the analyte by fragmentation lead to 10 or more fragment 3. After three cycles the number of variations of analyte are already 106.

FIG. 2 is a schematic depicting an example of a method in accordance with the principles described herein for the isolation of one or more variations of an analyte in a sample by binding specific variations of analyte to particle 8 (item 1) with attached analytical labels 9 (item 2) and attached affinity agents 10 (item 3) when incubated with a solution containing variations of analyte, such as antigens 11 (item 4). Particle with captured variations of analyte 12 (item 5) are isolated from bulk sample with intact analytical labels where multiple identical analytical labels are attached to particle by an X-Y bond and released by breaking the X-Y bond to free the analytical labels 13 (item 6) and allow detection and quantification of released analytical labels 14 (item 7) by comparison to a reference standard (item 8).

FIG. 3 is another schematic depicting an example of a method in accordance with the principles described herein directed to a method of isolation of all variations of analyte in a sample by binding all variation of analyte to a particle 16 (item 1) with attached analytical labels 17 (item 2) and unique attached affinity agents 18, 19, and 20 (items 3, 4 and 5) when incubated with a solution containing variations of analyte, such as antigens 21 (item 6). Particles with captured variations of analyte, such as antigens 22, 23, and 24 (items 7, 8 and 9) are isolated from bulk sample with intact analytical label where multiple identical analytical labels are attached to particle by an X-Y bond and released by breaking the X-Y bond to free the analytical labels 25 (item 10) and allow detection and quantification of released analytical labels 25 (item 10) by comparison to a reference standard 26 (item 11).

FIG. 4 is an additional schematic depicting an example of a method in accordance with the principles described herein of isolation of all variations of analyte in a sample by binding all variation of analyte to multiple particles 27 and 28 (item 1 and 2) with attached unique analytical labels 29 and 30 (items 3 and 4) and attached unique affinity agents 31 and 32 (items 5 and 6) when incubated with a solution containing variations of analyte, such as antigens 33 (item 7). Particles with captured variations of analyte, such as antigens 34 and 35 (items 8 and 9) are isolated from bulk sample with intact analytical label where multiple identical analytical labels are attached to a particle by an X-Y bond and released by breaking the X-Y bond to free the analytical labels 36 and 37 (item 10 and 11) and allow multiplexable detection and quantification of released analytical labels 36 and 37 (item 10 and 11) by comparison to with reference standard 38 (item 12).

### DETAILED DESCRIPTION OF SPECIFIC EMBODIMENTS

Methods, apparatus and kits in accordance with the invention described herein have application in the detection or isolation of rare molecules. Examples of such applications include, by way of illustration and not limitation, methods of isolation of variations of analyte by binding variations to a particle with attached analytical labels and separating the particles from the sample followed by removing analytical labels from particle and measurement of analyte molecules through measurement of analytical labels

Some examples in accordance with the invention described herein, are methods of isolation of variations of analyte molecules in a sample by binding variations to a particle through an affinity agent attached to particle which is also attached analytical labels and separating the particles from the sample followed by removal of analytical labels from the particles and measuring the analyte molecules through a measurement of analytical labels

Some examples in accordance with the invention described herein, are methods of isolation of variations of analyte molecules in a sample by binding variations to a particle through an affinity agent attached to particle by an X-Y bond which is also attached to analytical labels by an X-Y bond. Particles are separated from the sample after which analytical labels are released from the particle through a breakage of the X-Y bond connecting the analytical labels to the particle. A measurement of the released analytical label is then performed as a means of indirect measurement of analyte molecules.

Some examples in accordance with the invention are directed at the detection or isolation of variations of analytes which are cell free while other examples directed at the detection or isolation of variations of analyte that are cell bound or contained. Other examples are directed at the isolation and detection of variations of analyte that are included in rare cells which have been removed from the presence of non-rare cells. In some examples, rare cells are removed from the presence of non-rare cells by a porous matrix.

The term “variations of analyte” is a part, piece, fragment or modification of a molecule of biological or non-biological origin including small molecules like metabolites, co-factors, substrates, amino acids, metals, vitamins, fatty acids, biomolecules, peptides, carbohydrates or others, including macromolecules, like glycoconjugates, lipids, nucleic acids, polypeptides, receptors, enzymes, proteins as well as cells and tissues including cellular structures, peroxisomes, endoplasmic reticulum, endosomes, exosomes, lysosomes, mitochondria, cytoskeleton, membranes, nucleus, extra cellular matrix or other molecules typically measured.

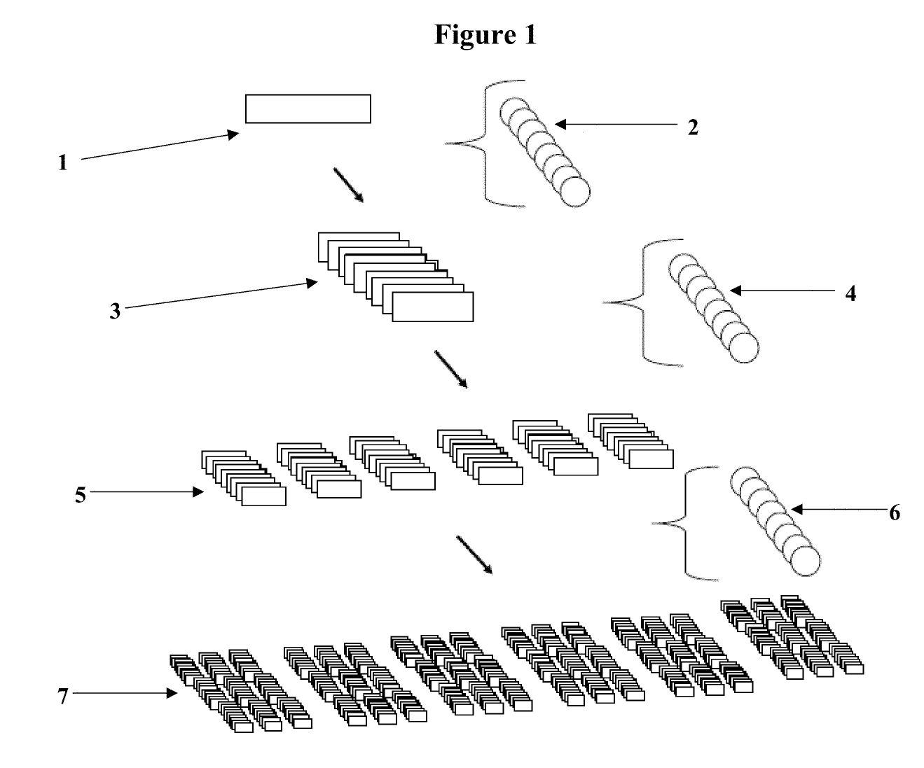

As explained above in brief description of the figures, FIG. 1 is a schematic depicting an example of the formation of “variations of analyte” by fragmentation, addition, or binding and shows an example of a group of proteases or peptidases acting on a single macromolecule such as a protein followed by additional reactions by a group of enzymes acting to create generated group of variations of the single protein. Variations of analyte can be generated from parts and pieces of cells and tissues as well as small molecules. Binding and association reactions also lead to additional differences in “variations of analyte” by generating bound forms which are variations that differ from unbound forms.

Some examples in accordance with the principles described herein are directed to methods of detecting one or more different populations of variations of analyte in a sample suspected of containing the one or more different populations of variations of analyte and non-analyte molecules. The term “variations of analyte” includes molecules but is not limited to biomolecules such as carbohydrates, lipids, nucleic acids, peptides and proteins. These variations of analyte can be used to measure enzymes, proteases, peptidase, proteins and inhibitors acting to form variations of analyte. These variations of analyte can be formed as natural or man-made origin, such as biological, therapeutics, or others. These variations of analyte can result intentionally from fragmentation, additions, binding or other modifications of analyte. Some examples in accordance with the principles described herein are directed to, addition of peptidases, enzymes, inhibitors or other reagents prior to the method of isolation such that variations of analyte are formed. These variations of analyte can be the result of intentional affinity reactions to isolate variations of analyte prior to analysis with the method.

The term “analytical label” refers to a chemical entity (organic or inorganic) which is capable of generating a signal detectable by optical, MS, or electrochemical means either directly on a porous matrix or in liquid. Analytical labels can be attached to an affinity agent specific for variations of an analyte, or attached to a label particle. Additionally, the analytical label can be released from an affinity agent or a label particle by breaking a chemical bond. The analytical label can be used to identify the affinity agent, particle labels, or variations of analyte. The analytical label can be used as an identifiable code for the affinity agent, label particle or variations of analyte (barcoding). In some examples the analytical label can be measured with an internal standard as a calibrator which is structurally similar or identical to the analytical label.

Some examples in accordance with the invention described herein are directed to methods of using mass labels as analytical labels for detection of variations of analyte. The term “mass label” refers to a molecule having a unique mass spectral signature that corresponds to, and is used to determine a presence and/or amount of rare molecules or affinity tag for rare molecules. The mass label can additionally be fluorescent, chemiluminescent or electrochemical in nature. The mass labels can, in some instances, be peptides with unique fragmentation patterns. The charges can be permanent or temporary charges.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “affinity agent” refers to a molecule capable of selectively binding to a specific molecule. The affinity agent can directly bind the variations of analyte of interest, or be directed to an affinity tag. Affinity agent can be attached to a capture particle or label particles or can bind a particle through electrostatic, hydrophobic, spatial, ionic or other interactions attracting the variations of analyte or an affinity tag to the affinity agent.

The term “label particle” refers to a particle bound to analytical label and affinity agents by a linkage. The term “capture particle” refers to a particle attached to an additional affinity agent or affinity tag by a linkage and may be used to capture the variation of analyte. The term “linkage,” refers to a bond between two groups which is denoted as an X-Y bond. The affinity agent is attached to the label particle by linkage which is an X-Y bond and analytical labels are attached to the label particle by linkage which is an X-Y bond. This linkage can be cleavable when subjected to certain conditions as described herein or permanent (does not undergo cleavage under conditions used). The term bond is typically a chemical bond i.e., a covalent bond or an ionic bond. Preferred linkages are covalent bond linkages.

Some examples in accordance with the invention described herein are directed to methods of measuring an analyte which use particle amplification of analytical labels through attachment of multiple analytic labels to a label particle. In some examples, directed to methods of amplification, there are multiple analytic labels attached to label particles with affinity agents. In other examples, additional affinity agents can be linked to capture particles and capture particles used to isolate label particles with affinity agents on to a porous matrix or magnet. Other examples in accordance with the principles described herein are directed to methods of binding and separation of variations of analyte where label particles and cells are isolated on porous matrix or magnetic particle and bound materials retained for analysis.

Examples in accordance with the invention described herein are directed to methods and kits for analysis. Other examples in accordance with the principles described herein are directed to apparatus for analysis.

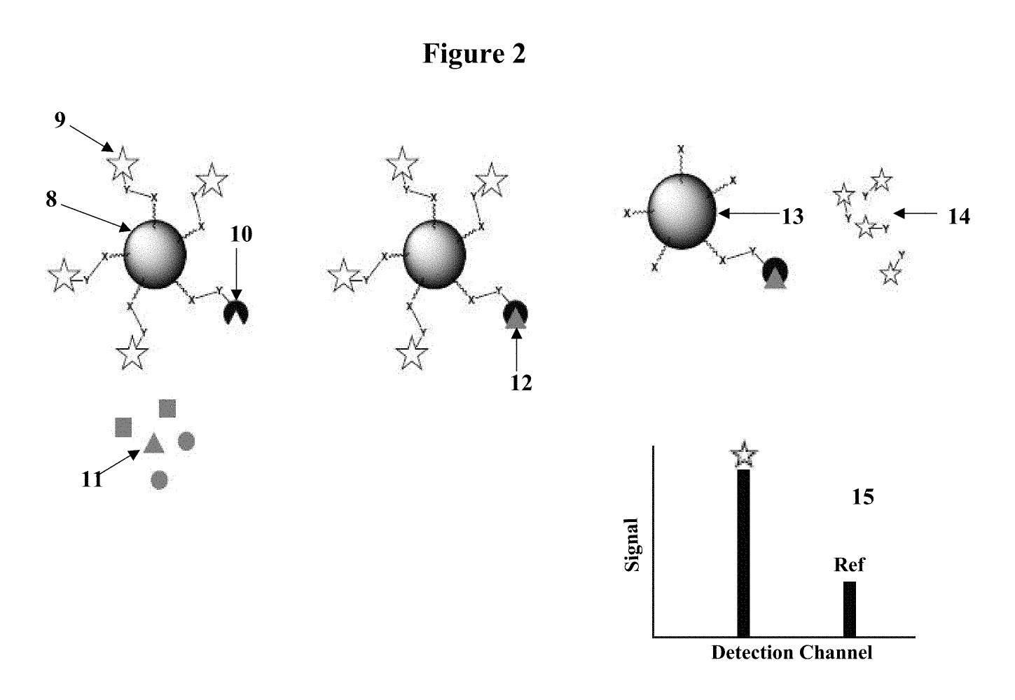

An example of a method, apparatus or kit for detection of a single variation of an analyte in accordance with the invention described herein is depicted in FIG. 2. As explained above in the brief description of FIG. 2, in this example the analytical label and affinity agent—which is capable of binding to a variation of the analyte—are attached through a linkage made between analytical labels on a label particle and a separate linkage between the affinity agent and the label particle. In the first step, the label particles with attached affinity agent are mixed with a sample containing a variation of the analyte. In a second step, the affinity agent binds to a variation of analyte and the label particle can be captured as is or bound by captured particles or cells and removed from samples by various means such as size exclusion filtration on a porous matrix, magnetic separation, or centrifugation. In this manner the variations of analyte bound to particles are separated from particles which are not bound to variations of an analyte. In a third step, label particles with captured variations of analyte, such as antigens, are subjected to conditions which release analytical labels from the label particle by breaking the X-Y bond and allow quantifiable detection of released analytical labels by comparison to a reference standard.

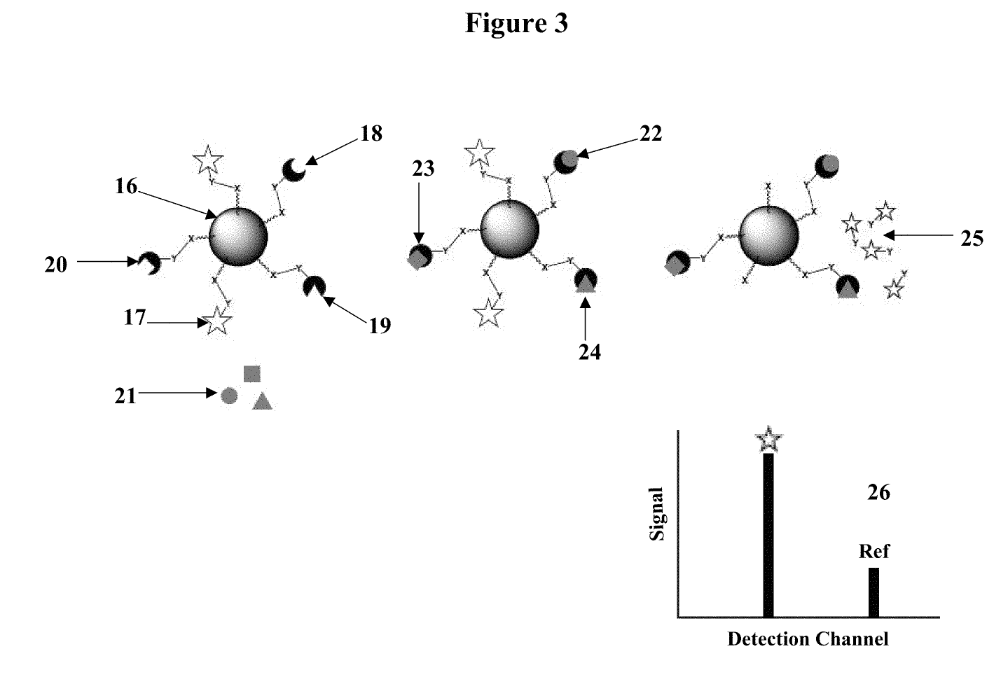

Another example of a method, apparatus or kit for detection of multiple variations of an analyte or analytes in accordance with the invention described herein is depicted in FIG. 3. As explained above in the description of FIG. 3, in this example the analytical label and multiple affinity agents—which are capable of binding to different variations of an analyte or analytes—are attached through a linkage made between analytical labels on a label particle and a separate linkage between the affinity agents and the label particle. In the first step, the label particles with attached affinity agents are mixed with a sample containing variations of an analyte or analytes. In a second step, the affinity agent binds to variations of analyte or analytes and the label particle can be captured as is, or bound by captured particles or cells and removed from samples by various means such as size exclusion filtration on a porous matrix, magnetic separation, or centrifugation. In this manner the variations of analyte or analytes bound to particles are separated from particles which are not bound to variations of an analyte or analytes. In a third step, label particles with captured variations of analyte or analytes, such as antigens, are subjected to conditions which release analytical labels from the label particle by breaking the X-Y bond and allow quantifiable detection of released analytical labels by comparison to a reference standard.

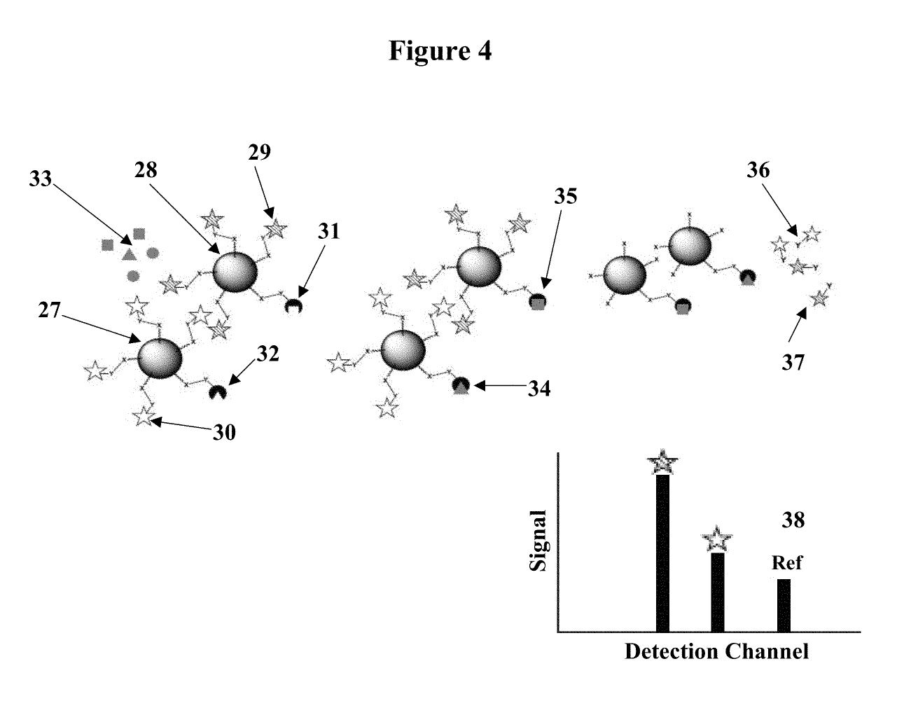

A further example of a method, apparatus or kit for analysis for detection of multiple variations of analyte or analytes in accordance with the invention described herein is depicted in FIG. 4. As explained above in the description of FIG. 4, there is shown an example of isolation of variations of analyte or analytes in a sample by binding with a label particle with an analytical label. In the first step, multiple label particles with unique attached affinity agents are mixed with a sample containing variations of an analyte or analytes. Multiple particles are used, each with a unique affinity agent and unique analytical label. In a second step, the affinity agent binds to variations of analyte or analytes and the label particle can be captured as is, or bound by captured particles or cells and removed from samples by various means such as size exclusion filtration on a porous matrix, magnetic separation, or centrifugation. In this manner the variations of analyte or analytes bound to particles are separated from particles which are not bound to variations of an analyte or analytes. In a third step, label particles with captured variations of analyte or analytes, such as antigens, are subjected to conditions which release analytical labels from the label particles by breaking the X-Y bond and allow quantifiable detection of multiple released analytical labels within the same sample by comparison to a reference standard.

### Examples of Variations of Analyte

In accordance with the principle described, “variations of analyte” can be derived from a molecule of biological or non-biological origin. The variations of analyte include but are not limited to biomolecules such as carbohydrates, lipids, nucleic acids, peptides and proteins. The variations of analyte can be the result of reactions, biological processes, disease, or intentional reactions and can be used to measure diseases or natural states. The variations of analyte can result from changes in molecules, such as proteins, enzymes, biologics or peptides, of man-made or natural origin and include bioactive and non-bioactive molecules such as those used in medical devices, therapeutic use, diagnostic use, used for measurement of processes, and those used as food, in agriculture, in production, as pro- or pre-biotics, in micro-organisms or cellular production, as chemicals for processes, for growth, measurement or control of cells, used for food safety and environmental assessment, used in veterinary products, and used in cosmetics.

The variations of analyte can be fragments of larger portions or bound forms and themselves can be used to measure other molecules, such as enzymes, peptidase and others. The measurements of other molecules, such as enzymes, peptidase and others can be based on formation of variations of analyte, such as enzymatic or proteolytic products. The measurements of other molecules, such as natural inhibitors, synthetic inhibitors and others, can be based on the lack of formation of variations of analyte.

The variations of analytes can be as the result of translation, or posttranslational modification by enzymatic or non-enzymatic modifications. Post-translational modification refers to the covalent modification of proteins during or after protein biosynthesis. Post-translational modification can be through enzymatic or non-enzymatic chemical reaction. Phosphorylation is a very common mechanism for regulating the activity of enzymes and is the most common post-translational modification. Enzymes can be oxidoreductases, hydrolases, lyases, isomerases, ligases or transferases as known commonly in enzyme taxonomy databases, such as http://enzyme.expasy.org/ or http://www.enzyme-database.org/ which have more than 6000 entries.

Common modification of variations of analyte include the addition of hydrophobic groups for membrane localization, addition of cofactors for enhanced enzymatic activity, diphthamide formation, hypusine formation, ethanolamine phosphoglycerol attachment, acylation, alkylation, amide bond formation such as amino acid addition or amidation, butyrylation gamma-carboxylation dependent on Vitamin K[15], glycosylation, the addition of a glycosyl group to either arginine, asparagine, cysteine, hydroxylysine, serine, threonine, tyrosine, or tryptophan resulting in a glycoprotein, malonylationhydroxylation, iodination, nucleotide addition such as ADP-ribosylation, phosphate ester (O-linked) or phosphoramidate (N-linked) formation such as phosphorylation or adenylylation, propionylation pyroglutamate formation, S-glutathionylation, S-nitrosylation S-sulfenylation (aka S-sulphenylation, succinylation or sulfation. Non-enzymatic modification include the attachment of sugars, carbamylation, carbonylation or intentional recombinate or synthetic conjugation such as biotinylation or addition of affinity agents, like histidine oxidation, formation of disulfide bonds between cystine residues, or pegylation (addition of polyethylene oxide groups).

Common reagents for intentional fragmentation and formation of variations of analytes such as peptides and proteins include peptidases or reagents know to react with peptides and proteins. Intentional fragmentation can generate specific fragments based on predicted cleavage sites for proteases (also termed peptidases or proteinases) and chemicals known to react with peptide and protein sequences. Common peptidases and chemicals for intentional fragmentation include Arg-C, Asp-N, BNPS oNCS/urea, caspase, chymotrypsin (low specificity), Clostripain, CNBr, enterokinase, factor Xa, formic acid, Glu-C, granzyme B, HRV3C protease, hydroxylamine, iodobenzoic acid, Lys-C, Lys-N, mild acid hydrolysis, NBS, NTCB, elastase, pepsin A, prolyl endopeptidase, proteinase K, TEV protease, thermolysin, thrombin, and trypsin. Common reagents for intentional inhibition of fragmentation include enzymes, peptidases, proteases, reductants, oxidants, chemical reactants, and chemical inhibitors for enzymes, peptidases, proteases including chemicals above listed.

### Examples of Breakable Linkage

In accordance with the invention, analytical labels and affinity agens are attached to label particles by linkages. Additionally, the analytical label is released from an affinity agent, or a label particle by breaking the linkage. The breakable linkage is defined as an “X-Y bond”. The phrase “X-Y bond” refers to a group of molecules allowing breakable connection of affinity agent or analytical label to a label particle. The phrase “X-Y bond” refers to a group of molecules having allowing linkage to be broken. The analytical labels contain an atom (Y) that link to an atom (X) on a label particle. The affinity agent can contain an atom (Y) that link to an atom (X) on a label particle. The X-Y bond may include sulfides, pyridyl disulfides, esters, ethers, thioesters, amides, thioamides, N-oxide, nitrogen-nitrogen, thioethers, peptides, carboxylates, chelates, guanidines, metals and so forth. The X-Y bond can be part of aliphatic hydrocarbon chains, polypeptides, polymers, aromatic hydrocarbons, aliphatic fatty acids, proteins, metals, carbohydrates, organic amines, ethers, esters, sulfides, phosphates, sulfates, nucleic acids, organic alcohols, and others (including mixtures of the above listed compounds) for example, whose structure can be varied by substitution, mass and chain length, for example. In the case of polymeric materials, the number of repeating units is adjusted in such a manner to optimize the reaction with the affinity agent or analytical labels. In some cases, the X-Y bond can be part of a long linker group to cause space between the affinity agent or analytical label and the label particle.

In some examples, the analytical label binding atom (Y) can be a thiol group which forms a bond to atom (X) which is also thiol group, such as those on alkyl groups, aromatic groups, peptides and proteins. In other examples the connecting disulfide bond can result from the reaction of a free thiol on the analytical label or affinity agent with a pyridyldithiol group present on the particle.

In some examples, the X and Y can be any combination of S, O, C, P, N, B, Si, Ni, Pd, Co, Ag, Fe, Cu, or Au. Functionalities present in the linking group may include esters, thioesters, amides, thioamides, ethers, guanidines, N-oxide, nitrogen-nitrogen, thioethers, carboxylate and so forth. In still other examples, the X or Y can be a metal binding molecule, such as a metal chelator attached to the affinity agent, analytical label or label particle which binds the metal, e.g. but not limited to proteins, peptides or molecules containing cysteine, histidine, arginine or tyrosine or thiol groups such as polyhistidine tag, polyagrinine tags, glutathione S-transferase (GST tag), immunoglobulin or many others.

In some cases affinity agents added to the label particles by the X-Y linkage group are affinity agents or affinity tags which bind one and another. Affinity tags and affinity agents pairs include but are not limited to biotin as affinity tags which binds to streptavidin or neutravidin as affinity agents, fluorescein which bind to anti-fluorescein antibodies as affinity agents. Affinity tags include other molecules which are bound by an antibody or protein and can serve as a binding partner to these affinity agents. In other examples, these affinity tags can be molecules which binds proteins that are not antibodies such as but not limited to, strep II tag peptides (peptide having SEQ ID NO:19 WSHPQFEK) which bind streptavidin-tactin protein, streptavidin-binding (SBP) peptide tag (peptide having SEQ ID NO:20 MDEKTTGWRG GHVVEGLAGE LEQLRARLEH HPQGQREP) which bind streptavidin protein, calmodulin-binding peptide (CBP) (peptide having SEQ ID NO:21 GVMPREETDSKTASPWKSAR) which bind calmodulin. In other examples affinity tags can be a carbohydrate molecule like amylose which binds to maltose-binding protein (MBP) (396 amino acid residues) as the affinity agent. In some case the affinity tags can be added to a second affinity agent such as biotin bound to an antibody which binds a variation of analyte. In this case the neutravidin is the affinity agent added to the label particles by the X-Y linkage and neutravidin binds the biotin which is bound to an antibody which can bind a variation of analyte.

In some cases the affinity tags can be directly attached to the variation of analyte. Examples include but are not limited to FLAG polypeptide tag (peptide having SEQ ID NO:22 DYKDDDDK), influenza hemagglutinin (HA) polypeptide tag (peptide having SEQ ID NO:23 YPYDVPDYA), c-Myc polypeptide tag (peptide having SEQ ID NO:24 EQKLISEEDL), S-tag polypeptide tag (peptide having SEQ ID NO:25 KETAAAKFERQHMDE), a puromycin which covalent links to a translated peptide or other molecules. These affinity tags with variation of analyte are bound by antibodies as affinity agents which are added to the label particles by the X-Y linkage group. In some cases, these affinity tags can be polypeptides which are fused to recombinant proteins during sub cloning of its cDNA or gene expression using various vectors for various host organisms (E. coli, yeast, insect, and mammalian cells). Additionally, the affinity tags can add properties to the analyte e.g. MBP and S-tag affinity tags increase the solubility of protein rare molecule and FLAG peptide tag can be cleaved with a specific protease, e.g. enterokinase (enteropeptidase).

### Examples of Analytical Labels

In some examples in accordance with the principles described herein, analytical labels are employed for detection and measurement of different populations of one or more variations of analyte in the methods, kits and apparatus. Analytical labels are molecules, metals, ions, atoms, or electrons that are detectable using an analytical method to yield information about the presence and amounts of one or more variations in the sample. The principles described herein are directed to methods using analytical labels of detecting one or more different variations of analyte in a sample suspected of containing one or more different populations of rare molecules and non-rare molecules. In some examples, the variations of analyte are in a cell or are of cellular origin. In other examples, the variations of analyte are free of cells or “cell free”. In other examples, the variation of analyte are cells. In some examples in accordance with the principles described herein, the concentration of the one or more different populations of variation of analyte is retained on the porous matrix or capture particle and reacted to generate an analytical label from the porous matrix or capture particle.

The analytical labels can be detected when retained on the porous matrix and released from the membrane into analysis liquid. The analytical labels can be detected when retained on the capture particle or cell and released from the capture particle or cell into analysis liquid. In some examples, the analytical labels are released from analytical label precursor into the analysis liquid without release of the variation of analyte. In other examples, the analytical labels are released from analytical label precursor into the analysis liquid with the variation of analyte also released. In other examples, the analytical labels are not released from analytical label precursor into the analysis liquid with the variation of analyte.

The porous matrix or analysis liquid can be subjected to analysis to determine the presence and/or amount of each different analytical label. The presence and/or amount of each different analytical label are related to the presence and/or amount of each different population of target rare molecules in the sample. The analytical labels can be measured by optical, electrochemical, or mass spectrographic methods as optical analytical labels, electrochemical analytical labels or mass spectrometry analytical labels (mass labels). The presence and/or amount of each different type of label, whether optical analytical labels, electrochemical analytical labels or mass spectrometry analytical labels can be related to each other to determine the presence and/or amount of each different population of target rare molecules retained on the porous substrate and/or capture particles.

In some examples, the analysis liquid with analytical labels can be transferred to a liquid receiving area that is sampled by an analyzer. In other examples, the analysis liquid with analytical labels can be retained on the porous matrix that is sampled by an analyzer. In other cases, the liquid receiving area can be inside an analyzer and the analysis liquid with analytical labels can be directly analyzed. In some analysis examples, the porous matrix is removed and placed in an analyzer where analysis of analytical labels is performed and converted to information about the presence and/or amount of each different variation of analyte or analytes.

In some methods in accordance with the invention described herein, analytical labels are generated by release from an analytical label precursor. In many examples, analytical labels can be generated after a reaction with a chemical to break a bond. In other examples, analytical labels are generated from analytical label precursor substrate such as chemical species that undergo reaction with an enzyme such as horseradish peroxidase, alkaline phosphatase, β-galactosidase, flavo-oxidase enzyme, urease or methyltransferase to name a few, to generate the label. In other examples, the analytical labels can be generated after reaction with an electron or ion, such as an electro-chemiluminescence (ECL) label.

As mentioned above, one or more linking groups X-Y are a cleavable moiety that is cleaved by a cleavage agent. The nature of the cleavage agent is dependent on the nature of the cleavable moiety. Cleavage of the cleavable moiety may be achieved by chemical or physical methods, involving one or more of oxidation, reduction, solvolysis, e.g., hydrolysis, photolysis, thermolysis, electrolysis, sonication, and chemical substitution, for example. Examples of cleavable moieties and corresponding cleavage agents, by way of illustration and not limitation, include disulfides that may be cleaved using a reducing agent, e.g., a thiol; diols that may be cleaved using an oxidation agent, e.g., periodate; diketones that may be cleaved by permanganate or osmium tetroxide; ether, esters, diazo linkages or oxime linkages that may be cleaved with hydrosulfite; β-sulfones, which may be cleaved under basic conditions; tetralkylammonium, trialkylsulfonium, tetralkylphosphonium, where the α-carbon is activated, e.g., with carbonyl or nitro, that may be cleaved with base; ester and thioester linkages that may be cleaved using a hydrolysis agent such as, e.g., hydroxylamine, ammonia or trialkylamine (e.g., trimethylamine or triethylamine) under alkaline conditions; quinones where elimination occurs with reduction; substituted benzyl ethers that can be cleaved photolytically; carbonates that can be cleaved thermally; metal chelates where the ligands can be displaced with a higher affinity ligand; thioethers that may be cleaved with singlet oxygen; hydrazone linkages that are cleavable under acidic conditions; quaternary ammonium salts (cleavable by, e.g., aqueous sodium hydroxide); trifluoroacetic acid-cleavable moieties such as, e.g., benzyl alcohol derivatives, teicoplanin aglycone, acetals and thioacetals; thioethers that may be cleaved using, e.g., HF or cresol; sulfonyls (cleavable by, e.g., trifluoromethane sulfonic acid, trifluoroacetic acid, or thioanisole); nucleophile-cleavable sites such as phthalamide (cleavable, e.g., with substituted hydrazines); ionic association (attraction of oppositely charged moieties) where cleavage may be realized by changing the ionic strength of the medium, adding a disruptive ionic substance, lowering or raising the pH, adding a surfactant, sonication, and/or adding charged chemicals; and photocleavable bonds that are cleavable with light having an appropriate wavelength such as, e.g., UV light at 300 nm or greater; for example.

In one example, a cleavable linkage may be formed using conjugation with N-succinimidyl 3-(2-pyridyldithio)propionate) (SPDP). For example, a label particle comprising an amine functionality is conjugated to SPDP and the resulting conjugate can then be reacted with a analytical label containing a thiol functionality, which results in the linkage of the mass label moiety to the conjugate. A disulfide reducing agent (such as, for example, dithiothreitol (DTT) or tris(2-carboxyethyl)phosphine (TCEP)) may be employed as a cleavage agent to release a thiolated peptide as an analytical label.

The phrase “optical analytical labels” refers to a group of molecules that allow for specific detection by optical means, such as: a chemiluminescent label like luminol, isoluminol, acridinium esters, adamantyl 1,2-dioxetane aryl phosphate, metals derivatives of or others commonly available to researchers in the field; a fluorescent label like fluorescein, lanthanide metals, Hoechst 33258, R-phycocyanin, B-phycoerythrin, R-phycoerythrin, rhodamine, DyLight dyes™, Texas red, metals or other list commonly available to researchers in the field (see http://www.fluorophores.org/) or; a chromogenic label such as tetramethylbenzidine (TMB), particles, metals or others. Optical analytical labels are detectable by optical methods like microscope, camera, optical reader, colorimeter, fluorometer, luminometer, reflectrometer, and others.

The phrase “electrochemical analytical labels” refers to potentiometric, capacitive and redox active compounds such as: metals like Pt, Ag, Pd, Au and many others or; particles like gold sols, graphene oxides and many others or; electron transport molecules like ferrocene, ferrocyanide, Os(VI)bipy and many others or; electrochemical redox active molecules like aromatic alcohols and amines such as 4-aminophenyl phosphate, 2-naphthol, para-nitrophenol phosphate; thiols or disulfides such as those on aromatics, aliphatics, amino acids, peptides and proteins; aromatic heterocyclic containing non-carbon ring atoms, like, oxygen, nitrogen, or sulfur such as like imidazoles, indoles, quinolones, thiazole, benzofuran and many others. Electrochemical analytical labels are detectable by impedance, capacitance, amperometry, electrochemical impedance spectroscopy and other measurement.

A label particle can include 1 to about 10⁸ analytical labels, or about 10 to about 10⁴ analytical labels, or about 10³ to about 10⁵ analytical labels, or about 10⁴ to about 10⁸ analytical labels, or about 10⁶ to about 10⁸ analytical labels, for example. The label particle can be comprised of proteins, polypeptides, polymers, particles, carbohydrates, nucleic acids, lipids or other macromolecules capable of forming bonds with analytical labels by attachment through the X-Y linkage. Multiple analytical labels on a single label particle allow amplification as every label particle can generate many analytical labels.

The phrase “mass labels” or “mass spectrometry analytical labels” refers to a group of molecules which generate unique mass spectroscopic signatures which corresponds to, and is used to determine a presence and/or amount of, each different variation of analyte or analytes. The mass labels are molecules of defined structure and molecular weight, which include but are not limited to, peptides, polymers, fatty acids, carbohydrates, organic amines, nucleic acids, and organic alcohols, for example. Molecular weight of mass labels can be varied by substitution and chain size, for example. In the case of polymeric materials, the number repeating units is adjusted such that the ion or ions formed from the mass label and detected by a mass spectrometer is in a region devoid of background interference.

A “mass label” is any molecule that results in a unique mass spectroscopic pattern when subjected to analysis by mass spectrometry. A “mass label precursor” is any molecule, particle, or combination of both from which a mass label may be formed or generated. The mass label precursor may, through the action of an alteration agent, be converted to a mass label by cleavage, by reaction with a moiety, by derivatization, or by addition or by subtraction of molecules, charges or atoms, for example, or a combination of two or more of the above.

The nature of the mass label precursors is dependent on one or more of the nature of the mass label, the nature of the MS method employed, the nature of the MS detector employed, the nature of the target rare molecules, the nature of the affinity agent, the nature of any immunoassay employed, the nature of the sample, the nature of any buffer employed, the nature of the separation, for example. In some examples, the mass label precursors are molecules whose mass can be varied by substitution and/or chain size. The mass labels produced from the mass label precursors are molecules of defined molecular weight and structure, which should not be present in the sample to be analyzed. Furthermore, the mass labels should be detectable by the MS detector and should not be subject to background interference by the sample or analysis liquid. Examples, by way of illustration and not limitation, of mass label precursors for use in methods in accordance with the principles described herein to produce mass labels include, by way of illustration and not limitation, polypeptides, organic and inorganic polymers, fatty acids, carbohydrates, cyclic hydrocarbons, aliphatic hydrocarbons, aromatic hydrocarbons, organic carboxylic acids, organic amines, nucleic acids, organic alcohols (e.g., alkyl alcohols, acyl alcohols, phenols, polyols (e.g., glycols), thiols, epoxides, primary, secondary and tertiary amines, indoles, tertiary and quaternary ammonium compounds, amino alcohols, amino thiols, phenolic amines, indole carboxylic acids, phenolic acids, vinylogous acid, carboxylic acid esters, phosphate esters, carboxylic acid amides, carboxylic acids from polyamides and polyesters, hydrazone, oxime, trimethylsilyl enol ether, acetal, ketal, carbamates, guanidines, isocyanates, sulfonic acids, sulfonamides, sulfonyl sulfates esters, monoglycerides, glycerol ethers, sphingosine bases, ceramines, cerebrosides, steroids, prostaglandins, carbohydrates, nucleosides and therapeutic drugs, for example.

Examples of peptides, which may function as mass labels, include, by way of illustration and not limitation, peptides that contain two or more of histidine, lysine, phenylalanine, leucine, alanine, methionine, asparagine, glutamine, aspartic acid, glutamic acid, tryptophan, proline, valine, tyrosine, glycine, threonine, serine, arginine, cysteine and isoleucine and derivatives thereof In some examples, the peptides have a molecular weight of about 100 to about 3,000 Da and may contain 3 to 30 amino acids, either naturally occurring or synthetic. The number of amino acids in the peptide is determined by, for example, the nature of the MS technique employed. For example, when using MALDI for detection, the peptide can have a mass in the range of about 600 to about 3,000 and is constructed of about 6 to about 30 amino acids. Alternatively, when using electrospray ionization for mass spectrometric analysis, the peptide has a mass in the range of about 100 to about 1,000 and is constructed of 1 to 30 amino acids or derivatives of, for example. In some examples, the number of amino acids in the peptide label may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, for example. The mass labels can include ionized groups, such as quaternary ammonium salts like carnitine, arginine salts, guanidine salts and their derivatives; quaternary aromatic ammonium salts like imidazole, pyrrole, histidine, quinoline, pyridine, indole, purine pyrimidine, and the like; tetra alkyl ammonium ions, tri alkyl sulfonium ions, tetra alkyl phosphonium ions and other examples

The use of peptides as mass labels has several advantages, which include, but are not limited to, the following: 1) relative ease of conjugation to proteins, antibodies, particles and other biochemical entities; 2) relative ease with which the mass can be altered to allow many different masses thus providing for multiplexed assay formats and standards; and 3) adjustability of the molecular weight for optimal performance with the mass spectrometer used for detection. For conjugation, the peptides can have a terminal cysteine that is employed in the conjugation. In order to aid in efficient ionization, the peptides can have permanently charged, or readily ionizable amine groups. In some examples, the peptides have N-terminal free amine and/or C-terminal free acid. In some examples, the peptides incorporate one or more stable isotopes or are derivatized with one or more stable isotopes. The peptides may be conjugated to a small molecule such as, for example, biotin or fluorescein, for binding to a corresponding binding partner for the small molecule, which in this example is streptavidin or antibody for fluorescein.

A polypeptide mass labels is any mass label that is composed of repeating units or sequences of amino acids. In the case of a polypeptide mass label, the identity and/or number of amino acid subunits can be adjusted to yield a mass label displaying a mass spectroscopic signature or peak not subject to background interference. Furthermore, mass spectrometry analytical labels may be produced from analytical label precursors having unique mass spectroscopic signatures, which are not present in the sample tested. The polypeptide analytical label precursors can include additional amino acids or derivatized amino acids, which allows for multiplexed measurements to obtain more than one result in a single analysis. Examples of polypeptide mass label precursors include, but are not limited to, polyglycine, polyalanine, polyserine, polythreonine, polycysteine, polyvaline, polyleucine, polyisoleucine, polymethionine, polyproline, polyphenylalanine, polytyrosine, polytryptophan, polyaspartic acid, polyglutamic acid, polyasparagine, polyglutamine, polyhistidine, polylysine and polyarginine, for example. In some examples, polypeptides are modified by catalysis. For example, by way of illustration and not limitation, phenol and aromatic amines can be added to polythreonine using a peroxidase enzyme as a catalyst. In another example, by way of illustration and not limitation, electrons can be transferred to aromatic amines using peroxidase enzyme as a catalyst. In another example, by way of illustration and not limitation, phosphates can be removed from organic phosphates using phosphatases as a catalyst.

In another example, a derivatization agent is employed to generate a mass label from a mass label precursor. For example, dinitrophenyl and other nitrophenyl derivatives may be formed from a mass label precursor. Other examples include, by way of illustration and not limitation, esterification, acylation, silylation, protective alkylation, derivatization by ketone-base condensations such as Schiff bases, cyclization, formation of fluorescent derivatives, and inorganic anions. The derivatization reactions can occur prior to MS analysis, after an affinity reaction or be used to generate mass label precursors which are conjugated to affinity reagents.

In some examples, the mass label precursor can include one or more isotopes such as, but not limited to, ²H, ¹³C, and ¹⁸O, for example, which remain in the mass label that is derived from the mass label precursor. The mass label can be detected based on a mass spectroscopic signature. In some examples, the mass label precursor is one that has a relatively high potential to cause a bond cleavage such as, but not limited to, alkylated amines, acetals, primary amines and amides, for example.

Internal standards are an important aspect of mass spectral analysis. In some examples, a second mass label or structurally similar compound is added to the analysis liquid (as an internal standard) which is used to quantify the mass label used for detection of the target rare molecule. In some instances the internal standard is isobaric (shares the same parent m/z as the mass label) but exhibits a unique mass spectroscopic pattern when fragmented inside the mass spectrometer. In other cases, the internal standard is selected such that the parent m/z differs slightly from that of the mass label. The internal standards may also contain additional amino acids or derivatized amino acids. Alternatively, the internal standard can be prepared by incorporating one or more isotopic elements such as, but not limited to ²H (D), ¹³C, and ¹⁸O, for example. In such a case the mass label (or internal standard) has a mass which differs from the naturally-occurring substance. For example, glycerol-C-d7, sodium acetate-C-d7, sodium pyruvate-C-d7, D-glucose-C-d7, deuterated glucose, and dextrose-C-d7, would serve as internal standards for glycerol, sodium acetate, sodium pyruvate, glucose and dextrose, respectively.

In some cases, internal standards and/or isobaric mass labels for multiplexed analyses make use of different peptides with amino acid substitutions such that the nominal molecular weight of the peptide mass labels remain unchanged while fragmentation inside the mass spectrometer results in unique mass spectroscopic signatures for the different mass label peptides. Examples of such peptides include, but is not limited to amino acid sequences of GAIIR and AAIVR which share a molecular weight of 528.7.7 Da, or RAAVIC and RGIAIC which share a molecular weight of 631.8 Da. In other cases, isobaric mass label peptides and internal standards make use of scrambled amino acid sequences such that fragmentation during mass spectrometric analysis produces one or more unique detectable fragments. Examples of mass label peptides with scrambled amino acid sequences that may be used as internal standards or multiplexable mass labels include but is not limited to amino acid sequences of GAIIR, AIIGR, and IGIAR, which all share a molecular weight of 527.7 Da.

Mass label peptides may be modified such that free amine groups (such as the N-terminal amine) or free carboxyl groups (such as the C-terminal carboxyl group) is altered to be a different functional group. By means of example and not limitation, free amines may be modified to be an acetyl group, formyl group, 9-fluorenylmethyloxycarbonyl (Fmoc), succinyl (Suc), chloroacetyl (Cl—Ac), maleimide (Mal), benzyloxycarbonyl (CBZ), bromoacetyl (Br—Ac), nitrilotriacetyl, terbutoxycarbonyl (Boc), 4-Hydroxyphenylpropionic acid (HPP), Lipoic acid (LA), pegylation, allyloxycarbonyl (Alloc), etc. Example of free carboxyl group modification include but is not limited to amidation (NH2), peptide aldehydes, alcohol peptide, chloromethylketone (CMK), 7-amino-4-methylcoumarin (AMC), p-nitroaniline (pNA), para-nitrophenol (—ONP), hydroxysucinimide ester (—OSu), etc. By way of example and not limitation, modifications to the free amines and/or carboxyl groups may be made for the purpose of increasing ionization efficiency, altering mass spectrometric patterns, generation of isobaric mass label peptides, to introduce functional groups that may be used to couple mass label peptides to label particles, or to alter the mass of the mass label peptide.

MS analysis determines the mass-to-charge ratio (m/z) of molecules for accurate identification and measurement. Generation of ions (ionization) may be accomplished by several techniques that include, but are not limited to, matrix-assisted laser desorption ionization (MALDI), atmospheric pressure chemical ionization (APCI), electrospray ionization (ESI), inductive electrospray ionization (iESI), chemical ionization (CI), electron impact ionization (EI), fast atom bombardment (FAB), field desorption/field ionization (FC/FI), thermospray ionization (TSP), and nanospray ionization, for example. The masses monitored by the mass spectrometer by several techniques that include, by way of illustration and not limitation, Time-of-Flight (TOF), ion traps, quadrupole mass filters, magnetic sectors, electric sectors, and Fourier transform ion cyclotron resonance (FTICR), for example. The MS method can be repeated in series (MSn), in which parent ions are selected and subjected to fragmentation, following which the fragments generated within the MS analyzer are measured. Fragments can be subjected to additional fragmentation within the MS analyzer for subsequent analysis. Sample processing steps are often performed before MS analysis, such as, by way of example and not limitation, liquid chromatography (LC), gas chromatography (GC), ion mobility spectrometer (IMS), and affinity separation.

Following the analysis by mass spectrometry, the presence and/or amount of each different mass label is related to the present and/or amount of each different population of target rare cells and/or the particle-bound target rare molecules. The relationship between the mass label and a target molecule is established through the use of an affinity agent, which is specific for the target molecule. Calibrators are employed to establish a relationship between an amount of signal from a mass label and an amount of target rare molecules in the sample.

### Examples of Affinity Agent

An affinity agent is a molecule capable selectively binding a target molecule. Selective binding involves the specific recognition of one of a molecule compared to substantially less recognition of other molecules. The terms “binding” or “bound” refers to the manner in which two moieties are associated to one another.

An affinity agent can be an immunoglobulin, protein, peptide, metal, carbohydrate, metal chelator, nucleic acid, or other molecule capable of binding selectively to a particular molecule. Selective binding involves the specific recognition of one of two different molecules for the other compared to substantially less recognition of other molecules. The association is through non-covalent binding such as a specific ionic binding, hydrophobic binding, pocket binding and the like. In contrast, “non-specific binding” may result from several factors including hydrophobic or electrostatic interactions between molecules that are general and not specific to any particular molecule in a class of similar molecules.

The affinity agents which are immunoglobulins may include complete antibodies or fragments thereof, including the various classes and isotypes, such as IgA, IgD, IgE, IgG1, IgG2a, IgG2b and IgG3, IgM, etc. Fragments thereof may include Fab, Fv and F(ab′)2, and Fab′, for example. In addition, aggregates, polymers, and conjugates of immunoglobulins or their fragments can be used where appropriate so long as binding affinity for a particular molecule is maintained.

Antibodies are specific for a rare molecule and can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography; and filtration, for example.

An affinity agent can additionally be a “cell affinity agent” capable of binding selectively to a rare molecule which is used for typing a rare cell or measuring a biological intracellular process of a cell. These affinity agents can be immunoglobulins that specifically recognize and bind to an antigen associated with a particular cell type and whereby the antigen is a component of the cell. The cell affinity agent is capable of being absorbed into or onto the cell. Selective cell binding typically involves “binding between molecules that is relatively dependent on specific structures of the binding pair (affinity agent and target rare molecule). Selective binding does not rely on non-specific recognition.

### Examples of Label and Capture Particles

Affinity agents can be attached to analytical labels and/or particles for the purpose of detection or isolation of rare molecules. This attachment can occur through “label particles” which are in-turn attached to analytical labels. Affinity agents can also be attached to “capture particles” which allow separation of bound and unbound analytical labels or rare molecules. The terms “attached” or “attachment” refers to the manner in which two moieties are connected. This can be accomplished by a direct bond between the two moieties or a linking group between the two moieties, covalent or otherwise. Alternatively, affinity agents can be attached to analytical labels and/or particles using additional “binding partners”. The phrase “binding partner” refers to a molecule that is a member of a specific binding pair of affinity agent or “affinity tags” that bind each respective partner other and not other molecules. In some examples, the affinity tags can be peptides, poly peptides or proteins such as polyhistidine tag, polyagrinine tags, glutathione S-transferase (GST tag), immunoglobulin or many others. In some cases, the affinity agent may be members of an immunological pair such as antigen to antibody or hapten to antibody, biotin to avidin, biotin to NeutrAvidin, biotin to streptavidin, IgG to protein A, secondary antibody to primary antibody, antibodies to fluorescent labels among others.

The “label particle” is a particulate material which is attached to the affinity agent through a linker arm or a binding pair. The “label particle” is capable of forming an X-Y cleavable linkage between the label particle and the analytical label as well as between the label particle and affinity agents or tags. The size of the label particle is large enough to accommodate one to 108 analytical labels and one to 108 affinity agents or tags. The ratio of analytical label and affinity agents or tags on a single label particle may be 108 to 1, 106 to 1, or 105 to 1, or 104 to 1, or 103 to 1, or 102 to 1, or 10 to 1, for example. The number of affinity agents or tags and analytical labels associated with the label particle is dependent on one or more of the nature and size of the affinity agent or tag, the nature and size of the label particle, the nature of the linker arm, the number and type of functional groups on the label particle, and the number and type of functional groups on the analytical label, for example.

The label particle can be used in combination with a capture particle where the capture particle is attached to an additional affinity agent specific to a particular variation of analyte. The “capture particle” and/or label particle is a particulate material which can be attached to the affinity agent or tag through a direct linkage or a binding pair. The composition of the label or capture particle entity may be organic or inorganic, magnetic or non-magnetic. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), dendrimer, melamine resin, nylon, poly(vinyl butyrate), for example, either used by themselves or in conjunction with other materials including latex. The particles may also be composed of carbon (e.g., carbon nanotubes), metal (e.g., gold, silver, and iron, including metal oxides thereof), colloids, dendrimers, dendrons, and liposomes, for example. In some examples, the particles can be silica.

In other some examples, particles can be magnetic. Particles may exhibit or be modified to exhibit free carboxylic acid, amine or tosyl groups, by way of example and not limitation. In some examples, particles can be mesoporous and include analytical labels within pores.

The diameter of the label or capture particle is dependent on one or more of the nature of the rare molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, the number, size, shape and molecular structure of associated label particles, for example. In some examples the average diameter of the capture particles is at least 1 μm but not more than about 20 μm.

The term “permeability” means the ability of a particles and molecule to diffuse through a barrier such as cellular walls or cellular membranes. In the case of rare molecule detection inside the cell, the diameter of the label particles must be small enough to allow the affinity agents (attached to the label particles) to enter the cell. Alternatively, the linkage between the label particle and the affinity agent must be of sufficient length and possess sufficient permeability to allow the affinity agent access to the interior of the cell. The label particle maybe coated with materials to increase “permeability” like collagenase, peptides, proteins, lipid, surfactants, and other chemicals known to increase particle permeability with respect to the cell.

When a porous matrix is employed in a filtration separation step, the diameter of the label particles must be small enough to efficiently pass through the pores of a porous matrix. Additionally, the diameter of the capture particles must be large enough to not pass through the pores of a porous matrix in order to retain the bound rare molecule on the matrix. In the case of cell-bound rare molecule detection, the cell must be of sufficient size to not pass through the pores of a porous matrix. In some examples in accordance with the principles described herein, the average diameter of the label particles should be at least 0.01 microns (10 nm) and not more than about 10 μm. In some examples, the adhesion of the particles to the surface is sufficiently strong such that the particle is retained on the porous matrix despite having a diameter smaller than the pores of the matrix.

The affinity agent can be prepared by direct attachment to the capture particles or label particles by linking groups. The linking group may also be a macro-molecule such as polysaccharides, peptides, proteins, nucleotides, and dendrimers. The linking groups may contain one or more cleavable or non-cleavable linking moieties. Cleavage of the cleavable moieties can be achieved through electrochemical reduction but also through chemical or physical methods. Such methods may involve furthers oxidation, reduction, solvolysis, e.g., hydrolysis, photolysis, thermolysis, electrolysis, sonication, and chemical substitution, for example. Photocleavable bonds that are cleavable with light having an appropriate wavelength e.g., UV light for example. The nature of the cleavage agent is dependent on the nature of the cleavable moiety.

The linking group between the particle and the affinity agent may be a chain of from 1 to about 200 or more atoms, each independently selected from the group normally consisting of hydrogen, carbon, oxygen, sulfur, nitrogen, and phosphorous, usually hydrogen, carbon and oxygen. The number of heteroatoms in the linking group may range from about 0 to about 8, from about 1 to about 6, or about 2 to about 4. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

Obtaining reproducibility in regards to the amounts of label and capture particles retained after separation and isolation is important for rare molecular analysis. Additionally, knowledge of the amounts of particles which enter a cell is important to maximize the amount of specific binding. Knowing the amount of particles which remain after washing is important to minimize the amount of non-selective binding. In order to make these determinations, it is helpful if the particles include “optical labels” which include fluorescent, colored, or chemiluminescence labels. Therefore, the presence of label particles can be measured by virtue of the presence of an optical label. The optical labels can be measured by microscopy and results compared for samples containing and lacking analyte. Fluorescent labels include but are not limited to dylight™, FITC, rhodamine compounds, phycoerythrin, phycocyanin, allophycocyanin, o-phthaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynylanthracene, squaraine dyes and fluorescamine, for example. A fluorescent microscope or fluorescent spectrometer may then be used to determine the location and amount of the label particles. Chemiluminescence labels examples include luminol, acridinium esters and acridinium sulfonamides to name a few. Colored labels include color particles, gold particles, enzymes which result in colorimetric reactions, to name a few.

### Examples of Porous Matrix and Filtration

In examples herein, porous matrices are used to isolate capture particles and cells during the isolation and/or detection of rare molecules. Porous matrices are used where the particles are sufficiently smaller than the pore size of the matrix such that physically the particles can pass through the pores. In other examples, the particles are sufficiently larger than the pore size of the matrix such that physically the particles cannot pass through the pores.

In some methods in accordance with the principles described herein, the sample is incubated with an affinity agent consisting of an analytical label and label particle, for each different population of rare molecules. The affinity agent comprises a specific binding partner that is specific for and binds to a rare molecule of one of the populations of the rare molecules. The rare molecules can be cell bound or cell free. The affinity agent with analytical label and label particle are retained on the surface of a membrane.

In some examples the porous matrix used for filtration is such that the pores are of sufficient size to allow unbound label particles to pass through the pores while cells comprising rare molecules are retained on the porous matrix along with label particles which are bound to said cells. In still other methods, affinity agents on label particles can be additionally bound through “binding partners” or “sandwich assays” to capture particles (e.g. magnetic particles) or to a surface. In the prior case, the capture particles are retained on the surface of the porous matrix.

In some examples, the concentration of the one or more different populations of rare molecules is enhanced over that of the non-rare molecules to form a concentrated sample. In some examples, the sample is subjected to a filtration procedure using a porous matrix that retains the rare molecules while allowing the non-rare molecules to pass through the porous matrix thereby increasing the concentration of the rare molecules. In the event that one or more rare molecules are non-cellular, i.e., not associated with a cell or other biological particle, the sample is combined with one or more capture particle entities wherein each capture particle entity comprises a binding partner for the non-cellular rare molecule of each of the populations of non-cellular rare molecules to render the non-cellular rare molecules in particulate form, i.e., to form particle-bound non-cellular rare molecules. The combination of the sample and the capture particle entities is held for a period of time and at a temperature which permits the binding of non-cellular rare molecules with corresponding binding partners of the capture particle entities. A pressure gradient (i.e. vacuum) is applied to the sample on the porous matrix to facilitate passage of non-rare cells, non-rare molecules, and other sample contents through the matrix. The pressure gradient applied is dependent on one or more of the nature and size of the different populations of rare cells and/or particle reagents, the nature of the porous matrix, and the size of the pores of the porous matrix, for example.

Contact of the sample with the porous matrix is continued for a period of time sufficient to achieve retention of cellular rare molecules and/or particle-bound non-cellular rare molecules on a surface as discussed above. The period of time is dependent on one or more of the nature and size of the different populations of rare cells and/or particle-bound rare molecules, the nature of the porous matrix, the size of the pores of the porous matrix, the level of vacuum applied to the blood sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

An amount of each different affinity agent that is employed in the methods in accordance with the principles described herein is dependent on one or more of the nature and potential amount of each different population of rare molecule, the nature of the analytical label, the natured of attachment, the nature of the affinity agent, the nature of a cell if present, the nature of a particle if employed, and the amount and nature of a blocking agent if employed, for example. In some examples, the amount of each different modified affinity agent employed is about 0.001 μg/μL to about 100 μg/μL, or about 0.001 μg/μL to about 80 μg/μL, or about 0.001 μg/μL to about 60 μg/μL, or about 0.001 μg/μL to about 40 μg/μL, or about 0.001 μg/μL to about 20 μg/μL, or about 0.001 μg/μL to about 10 μg/μL, or about 0.5 μg/μL to about 100 μg/μL, or about 0.5 μg/μL to about 80 μg/μL, or about 0.5 μg/μL to about 60 μg/μL, or about 0.5 μg/μL to about 40 μg/μL, or about 0.5 μg/μL to about 20 μg/μL, or about 0.5 μg/μL to about 10 μg/μL, for example.

The porous matrix is a solid or semi-solid material, which is impermeable to liquid (except through one or more pores of the matrix) in accordance with the invention described herein. The porous matrix is associated with a porous matrix holder and a liquid holding well. The association between porous matrix and holder can be achieved with the use of an adhesive. The association between porous matrix in the holder and the liquid holding well can be through direct contact or with a flexible gasket surface.

The porous matrix is a solid or semi-solid material and may be comprised of an organic or inorganic, water insoluble material. The porous matrix is non-bibulous, which means that the membrane is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the membrane is at least 95% free of fibers, or at least 99% free of fibers, or at least 99.5%, or at least 99.9% free of fibers, or 100% free of fibers.

The porous matrix can have any of a number of shapes such as, for example, planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the nature or shape of holder for the membrane, of the microfluidic surface, of the liquid holding well for example. In some examples the shape of the porous matrix is circular, oval, rectangular, square, track-etched, planar or flat surface (e.g., strip, disk, film, membrane, and plate), for example.

The porous matrix and holder may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly (vinyl chloride), polyacrylamide, polyacrylate, polyethylene, polypropylene, poly-(4-methylbutene), polystyrene, polymethacrylate, poly-(ethylene terephthalate), nylon, poly(vinyl butyrate), poly(chlorotrifluoroethylene), poly(vinylbutyrate), polyimide, polyurethane, and paraylene; silanes; silicon; silicon nitride; graphite; ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and Pyrex®); and bioresorbable polymers (such as, e.g., polylactic acid, polycaprolactone and polyglycoic acid); for example, either used by themselves or in conjunction with one another and/or with other materials. The material for fabrication of the porous matrix and holder are non-bibulous and does not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The material for fabrication of the porous matrix and holder may be the same or different materials.

The porous matrix for each liquid holding well comprises at least one pore and no more than about 2,000,000 pores per square centimeter (cm²). In some examples the number of pores of the porous matrix per cm² is 1 to about 2,000,000, or 1 to about 1,000,000, or 1 to about 500,000, or 1 to about 200,000, or 1 to about 100,000, or 1 to about 50,000, or 1 to about 25,000, or 1 to about 10,000, or 1 to about 5,000, or 1 to about 1,000, or 1 to about 500, or 1 to about 200, or 1 to about 100, or 1 to about 50, or 1 to about 20, or 1 to about 10, or 2 to about 500,000, or 2 to about 200,000, or 2 to about 100,000, or 2 to about 50,000, or 2 to about 25,000, or 2 to about 10,000, or 2 to about 5,000, or 2 to about 1,000, or 2 to about 500, or 2 to about 200, or 2 to about 100, or 2 to about 50, or 2 to about 20, or 2 to about 10, or 5 to about 200,000, or 5 to about 100,000, or 5 to about 50,000, or 5 to about 25,000, or 5 to about 10,000, or 5 to about 5,000, or 5 to about 1,000, or 5 to about 500, or 5 to about 200, or 5 to about 100, or 5 to about 50, or 5 to about 20, or 5 to about 10, for example. The density of pores in the porous matrix is about 1% to about 20%, or about 1% to about 10%, or about 1% to about 5%, or about 5% to about 20%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein. The size of the pores of the porous matrix is dependent on the nature of the liquid, the size of the cell, the size of the capture particle, the size of analytical label, the size of an analyte, the size of label particles, the size of non-rare molecules, and the size of non-rare cells, for example. In some examples the average size of the pores of the porous matrices is about 0.1 to about 20 microns, or about 0.1 to about 5 microns, or about 0.1 to about 1 micron, or about 1 to about 20 microns, or about 1 to about 5 microns, or about 1 to about 2 microns, or about 5 to about 20 microns, or about 5 to about 10 microns, for example.

Pores within the matrix may be fabricated in accordance with the principles described herein, for example, by microelectromechanical (MEMS) technology, metal oxide semi-conductor (CMOS) technology, micro-manufacturing processes for producing microsieves, laser technology, irradiation, molding, and micromachining, for example, or a combination thereof.

In some cases, the porous matrix is permanently attached to a holder which can be associated to the bottom of the liquid holding well and to the top of the vacuum manifold where the porous matrix is positioned such that liquid can flow from liquid holding well to vacuum manifold. In some examples, the porous matrix in the holder can be associated to a microfluidic surface, top cover surface and/or bottom cover surface. The holder may be constructed of any suitable material that is compatible with the material of the porous matrix. Examples of such materials include, by way of example and not limitation, any of the materials listed above for the porous matrix. The material for the housing and for the porous matrix may be the same or may be different. The holder may also be constructed of non-porous glass or plastic film.

Examples of plastic film materials include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently fixed attached to a holder by adhesion using thermal bonding, mechanical fastening or through use of permanently adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein, lignin. The plastic film or the adhesive can be electrically conductive materials and the conductive material coatings or materials can be patterned across specific regions of the holder surface.

The porous matrix in the holder is generally part of a filtration module where the porous matrix is part of an assembly for convenient use during filtration. The holder has a surface which facilitates contact with associated surfaces but is not permanently fixed attached to these surfaces and can be removed. A top gasket maybe applied to the removable holder between the liquid holding wells. A bottom gasket maybe applied to the removable holder between the manifold for vacuum. A gasket is a flexible material that facilitates a liquid or air impermeable seal upon compression. The holder maybe constructed of gasket material. Examples of gasket shapes include flat, embossed, patterned, or molded sheets, rings, circles, ovals, with cut out areas to allow sample to flow from porous matrix to vacuum manifold. Examples of gasket materials include paper, rubber, silicone, metal, cork, felt, neoprene, nitrile rubber, fiberglass, polytetrafluoroethylene like PTFE or Teflon or a plastic polymer like polychlorotrifluoroethylene.

In some examples, vacuum is applied to the concentrated and treated sample on the porous matrix to facilitate passage of non-rare cells through the matrix. The level of vacuum applied is dependent on one or more of the nature and size of the different populations of biological particles, the nature of the porous matrix, and the size of the pores of the porous matrix, for example. In some examples, the level of vacuum applied is about 1 millibar to about 100 millibar, or about 1 millibar to about 80 millibar, or about 1 millibar to about 50 millibar, or about 1 millibar to about 40 millibar, or about 1 millibar to about 30 millibar, or about 1 millibar to about 25 millibar, or about 1 millibar to about 20 millibar, or about 1 millibar to about 15 millibar, or about 1 millibar to about 10 millibar, or about 5 millibar to about 80 millibar, or about 5 millibar to about 50 millibar, or about 5 millibar to about 30 millibar, or about 5 millibar to about 25 millibar, or about 5 millibar to about 20 millibar, or about 5 millibar to about 15 millibar, or about 5 millibar to about 10 millibar, for example. In some examples the vacuum is an oscillating vacuum, which means that the vacuum is applied intermittently at regular or irregular intervals, which may be, for example, about 1 second to about 600 seconds, or about 1 second to about 500 seconds, or about 1 second to about 250 seconds, or about 1 second to about 100 seconds, or about 1 second to about 50 seconds, or about 10 seconds to about 600 seconds, or about 10 seconds to about 500 seconds, or about 10 seconds to about 250 seconds, or about 10 seconds to about 100 seconds, or about 10 seconds to about 50 seconds, or about 100 seconds to about 600 seconds, or about 100 seconds to about 500 seconds, or about 100 seconds to about 250 seconds, for example. In this approach, vacuum is oscillated at about 0 millibar to about 10 millibar, or about 1 millibar to about 10 millibar, or about 1 millibar to about 7.5 millibar, or about 1 millibar to about 5.0 millibar, or about 1 millibar to about 2.5 millibar, for example, during some or all of the application of vacuum to the blood sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

### Examples of Rare Molecules

The phrase “rare molecules” refers to molecules that may be detected as analytes in a sample. One or more variations of analytes are indicative of particular populations of rare molecules. The phrase “population of molecules” refers to a group of rare molecules that share a common portion of molecular structure that specifically defines a group of rare molecules. The phrase “specific for” means that the common rare molecules distinguishes the group of rare molecules from other molecules.

The phrase “cell free rare molecules” refers to rare molecules that are not bound to a cell and/or that freely circulate in a sample. Such non-cellular rare molecules include biomolecules useful in medical diagnosis and treatments of diseases. Medical diagnosis of diseases include, but are not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, metabolic disease, diabetes, liver diseases, infectious diseases and other biomolecules useful in medical diagnosis of diseases, for example.

The following are non-limiting examples of samples that rare molecules that can be measured in. The sample to be analyzed is one that is suspected of containing rare molecules. The samples may be biological samples or non-biological samples. Biological samples may be from a plant, animal, protists or other living organism including Animalia, fungi, plantae, chromista, or protozoa or other eukaryote species or bacteria, archaea, or other prokaryote species. Non-biological samples include aqueous solutions, environmental, products, chemical reaction production, waste streams, foods, feed stocks, fertilizers, fuels, and the like. Biological samples include biological fluids such as whole blood, serum, plasma, sputum, lymphatic fluid, semen, exosome, lipids, vaginal mucus, feces, urine, spinal fluid, saliva, stool, cerebral spinal fluid, tears, mucus, or tissues for example. Biological tissue includes, by way of illustration, hair, skin, sections or excised tissues from organs or other body parts, for example rare molecules may be from tissues, for example, lung, bronchus, colon, rectum, extra cellular matrix, dermal, vascular, stem, lead, root, seed, flower, pancreas, prostate, breast, liver, bile duct, bladder, ovary, brain, central nervous system, kidney, pelvis, uterine corpus, oral cavity or pharynx or cancers. In many instances, the sample is aqueous such as a urine, whole blood, plasma or serum sample, in other instances the sample must be made into a solution or suspension for testing.

The sample can be one that contains cells such as, for example, non-rare cells and rare cells where rare molecules are detected from the rare cells. The rare molecules from cells may be from any organism, and are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In other examples of methods in accordance with the invention described herein, the sample to be tested is a blood sample from an organism such as, but not limited to, a plant or animal subject, for example. In some examples of methods in accordance with the principles described herein, the sample to be tested is a sample from an organism such as, but not limited to, a mammal subject, for example. Cells with rare molecules may be from a tissue of mammal, for example, lung, bronchus, colon, rectum, pancreas, prostate, breast, liver, bile duct, bladder, ovary, brain, central nervous system, kidney, pelvis, uterine corpus, oral cavity or pharynx or cancers.

Rare molecule fragments can be used to measure peptidases of interest including those in the MEROPS is an on-line database for peptidases (also known as proteases) and total ˜902212 different sequences of aspartic, cysteine, glutamic, metallo, asparagine, serine, threonine and general peptidases catalytics types which are further categorized and include those listed for the following pathways: 2-Oxocarboxylic acid metabolism, ABC transporters, African trypanosomiasis, alanine, aspartate and glutamate metabolism, allograft rejection, Alzheimer's disease, amino sugar and nucleotide sugar metabolism, amoebiasis, AMPK signaling pathway, amyotrophic lateral sclerosis (ALS), antigen processing and presentation, apoptosis, arachidonic acid metabolism, arginine and proline metabolism, arrhythmogenic right ventricular cardiomyopathy (ARVC), asthma, autoimmune thyroid disease, B cell receptor signaling pathway, bacterial secretion system, basal transcription factors, beta-alanine metabolism, bile secretion, biosynthesis of amino acids, biosynthesis of secondary metabolites, biosynthesis of unsaturated fatty acids, biotin metabolism, bisphenol degradation, bladder cancer, cAMP signaling pathway, carbon metabolism, cardiac muscle contraction, cell adhesion molecules (CAMs), cell cycle, cell cycle—yeast, chagas disease (American trypanosomiasis), chemical carcinogenesis, cholinergic synapse, colorectal cancer, complement and coagulation cascades, cyanoamino acid metabolism, cysteine and methionine metabolism, cytokine-cytokine receptor interaction, cytosolic DNA-sensing pathway, degradation of aromatic compounds, dilated cardiomyopathy, dioxin degradation, DNA replication, dorso-ventral axis formation, drug metabolism—other enzymes, endocrine and other factor-regulated calcium reabsorption, endocytosis, epithelial cell signaling in helicobacter pylori infection, Epstein-Barr virus infection, estrogen signaling pathway, Fanconi anemia pathway, fatty acid elongation, focal adhesion, folate biosynthesis, foxO signaling pathway, glutathione metabolism, glycerolipid metabolism, glycerophospholipid metabolism, glycosylphosphatidylinositol(GPI)-anchor bio-synthesis, glyoxylate and dicarboxylate metabolism, GnRH signaling pathway, graft-versus-host disease, hedgehog signaling pathway, hematopoietic cell lineage, hepatitis B, herpes simplex infection, HIF-1 signaling pathway, hippo signaling pathway, histidine metabolism, homologous recombination, HTLV-I infection, huntington's disease, hypertrophic cardiomyopathy (HCM), influenza A, insulin signaling pathway, legionellosis, Leishmaniasis, leukocyte transendothelial migration, lysine biosynthesis, lysosome, malaria, MAPK signaling pathway, meiosis—yeast, melanoma, metabolic pathways, metabolism of xenobiotics by cytochrome P450, microbial metabolism in diverse environments, microRNAs in cancer, mineral absorption, mismatch repair, natural killer cell mediated cytotoxicity, neuroactive ligand-receptor interaction, NF-kappa B signaling pathway, nitrogen metabolism, NOD-like receptor signaling pathway, non-alcoholic fatty liver disease (NAFLD), notch signaling pathway, olfactory transduction, oocyte meiosis, osteoclast differentiation, other glycan degradation, ovarian steroidogenesis, oxidative phosphorylation, p53 signaling pathway, pancreatic secretion, pantothenate and CoA biosynthesis, parkinson's disease, pathways in cancer, penicillin and cephalosporin biosynthesis, peptidoglycan biosynthesis, peroxisome, pertussis, phagosome, phenylalanine metabolism, phenylalanine, tyrosine and tryptophan biosynthesis, phenylpropanoid biosynthesis, PI3K-Akt signaling pathway, plant-pathogen interaction, platelet activation, PPAR signaling pathway, prion diseases, proteasome, protein digestion and absorption, protein export, protein processing in endoplasmic reticulum, proteoglycans in cancer, purine metabolism, pyrimidine metabolism, pyruvate metabolism, Rapt signaling pathway, Ras signaling pathway, regulation of actin cyto-skeleton, regulation of autophagy, renal cell carcinoma, renin-angiotensin system, retrograde endocannabinoid signaling, rheumatoid arthritis, RIG-I-like receptor signalling pathway, RNA degradation, RNA transport, salivary secretion, salmonella infection, serotonergic synapse, small cell lung cancer, spliceosome, staphylococcus aureus infection, systemic lupus erythematosus, T cell receptor signaling pathway, taurine and hypotaurine metabolism, terpenoid backbone bio-synthesis, TGF-beta signaling pathway, TNF signaling pathway, Toll-like receptor signaling pathway, toxoplasmosis, transcriptional misregulation in cancer, tryptophan metabolism, tuberculosis, two-component system, type I diabetes mellitus, ubiquinone and other terpenoid-quinone biosynthesis, ubiquitin mediated proteolysis, vancomycin resistance, viral carcinogenesis, viral myocarditis, vitamin digestion and absorption Wnt signaling pathway.

Rare molecule fragments that can be used to measure peptidase inhibitors of interest included those in the MEROPS (an on-line database for peptidase inhibitors) which include a total of ˜133535 different sequences of where a family is a set of homologous peptidase inhibitors with a homology. The homology is shown by a significant similarity in amino acid sequence either to the type inhibitor of the family, or to another protein that has already been shown to be homologous to the type inhibitor, and thus a member of. The reference organism for the family is shown ovomucoid inhibitor unit 3 (Meleagris gallopavo) aprotinin (Bos taurus), soybean Kunitz trypsin inhibitor (Glycine max), proteinase inhibitor B (Sagittaria sagittifolia), alpha-1-peptidase inhibitor (Homo sapiens), ascidian trypsin inhibitor (Halocynthia roretzi), ragi seed trypsin/alpha-amylase inhibitor (Eleusine coracana), trypsin inhibitor MCTI-1 (Momordica charantia), Bombyx subtilisin inhibitor (Bombyx mori), peptidase B inhibitor (Saccharomyces cerevisiae), marinostatin (Alteromonas sp.), ecotin (Escherichia coli), Bowman-Birk inhibitor unit 1 (Glycine max), eglin c (Hirudo medicinalis), hirudin (Hirudo medicinalis), antistasin inhibitor unit 1 (Haementeria officinalis), streptomyces subtilisin inhibitor (Streptomyces albogriseolus), secretory leukocyte peptidase inhibitor domain 2 (Homo sapiens), mustard trypsin inhibitor-2 (Sinapis alba), peptidase inhibitor LMPI inhibitor unit 1 (Locusta migratoria), potato peptidase inhibitor II inhibitor unit 1 (Solanum tuberosum), secretogranin V (Homo sapiens), BsuPI peptidase inhibitor (Bacillus subtilis), pinA Lon peptidase inhibitor (Enterobacteria phage T4), cystatin A (Homo sapiens), ovocystatin (Gallus gallus), metallopeptidase inhibitor (Bothrops jararaca), calpastatin inhibitor unit 1 (Homo sapiens), cytotoxic T-lymphocyte antigen-2 alpha (Mus musculus), equistatin inhibitor unit 1 (Actinia equina), survivin (Homo sapiens), aspin (Ascaris suum), saccharopepsin inhibitor (Saccharomyces cerevisiae), timp-1 (Homo sapiens), Streptomyces metallopeptidase inhibitor (Streptomyces nigrescens), potato metallocarboxypeptidase inhibitor (Solanum tuberosum), metallopeptidase inhibitor (Dickeya chrysanthemi), alpha-2-macroglobulin (Homo sapiens), chagasin (Leishmania major), oprin (Didelphis marsupialis), metallocarboxypeptidase A inhibitor (Ascaris suum), leech metallocarboxypeptidase inhibitor (Hirudo medicinalis), latexin (Homo sapiens), clitocypin (Lepista nebularis), proSAAS (Homo sapiens), baculovirus P35 caspase inhibitor (Spodoptera litura nucleopolyhedrovirus), p35 homologue (Amsacta moorei entomopoxvirus), serine carboxypeptidase Y inhibitor (Saccharomyces cerevisiae), tick anticoagulant peptide (Ornithodoros moubata), madanin 1 (Haemaphysalis longicornis), squash aspartic peptidase inhibitor (Cucumis sativus), staphostatin B (Staphylococcus aureus), staphostatin A (Staphylococcus aureus), triabin (Triatoma pallidipennis), pro-eosinophil major basic protein (Homo sapiens), thrombostasin (Haematobia irritans), Lentinus peptidase inhibitor (Lentinula edodes), bromein (Ananas comosus), tick carboxypeptidase inhibitor (Rhipicephalus bursa), streptopain inhibitor (Streptococcus pyogenes), falstatin (Plasmodium falciparum), chimadanin (Haemaphysalis longicornis), {Veronica} trypsin inhibitor (Veronica hederifolia), variegin (Amblyomma variegatum), bacteriophage lambda CIII protein (bacteriophage lambda), thrombin inhibitor (Glossina morsitans), anophelin (Anopheles albimanus), Aspergillus elastase inhibitor (Aspergillus fumigatus), AVR2 protein (Passalora fulva), IseA protein (Bacillus subtilis), toxostatin-1 (Toxoplasma gondii), AmFPI-1 (Antheraea mylitta), cvSI-2 (Crassostrea virginica), macrocypin 1 (Macrolepiota procera), HflC (Escherichia coli), oryctin (Oryctes rhinoceros), trypsin inhibitor (Mirabilis jalapa), F1L protein (Vaccinia virus), NvCI carboxypeptidase inhibitor (Nerita versicolor), Sizzled protein (Xenopus laevis), EAPH2 protein (Staphylococcus aureus), and Bowman-Birk-like trypsin inhibitor (Odorrana versabilis). Rare molecule fragments can be used to measure synthetic inhibition of peptidase inhibitors. The afore mentioned data base also includes examples thousands of different small molecule inhibitors that can mimic the inhibitory properties for any member or the above listed family.

Rare molecules of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemokine (C—C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin, LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metalloproteinase, MPO Myeloperoxidase, mTOR Mammalian of rapamycin, NADH Nicotinamide adenine di-nucleotide, NGF Nerve growth factor, NFκB Nuclear factor kappa-light-chain-enhancer of activated B cells, NGAL Neutrophil gelatinase lipocalin, NOS Nitric oxide synthase NOX NADPH oxidase NPY Neuropeptide Yglucose, insulin, proinsulin, c peptide OHdG Hydroxy-deoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidyl-serine, PR Protienase, PYY Neuropeptide like peptide Y, RAGE Receptors for AGE, ROS Reactive oxygen species, S100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factor-β, TIMP Tissue inhibitor of metalloproteinases, TNF α Tumor necrosis factors-α, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare molecules of interest that are highly expressed by pancreatic tissue or found in the pancreas include insulin, proinsulin, c-peptide, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymotrypsinogen B1 Intracellular, CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), PDX1 insulin promoter factor 1, MAFA Maf family of transcription factors, GLUT2 Glucose Transporter Type 2, ST8SIA1 Alpha-N-acetylneuraminide alpha-2,8-sialyltransferase, CD9 tetraspanin, ALDH1A3 aldehyde dehydrogenase, CTFR cystic fibrosis transmembrane conductance regulator as well as diabetic auto immune antibodies such as against GAD, IA-2, IAA, ZnT8 or the like.

Rare molecule fragments include those of insulin, pro-insulin or c peptide generated by the following peptidases known to naturally act on insulin; archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, matrix metallopeptidase-9 and others. These fragments include but are not limited to the following sequences of SEQ ID NO:1 MALWMRLLPLLALLALWGP, SEQ ID NO:2 MALWMRLLPL, SEQ ID NO:3 ALLALWGPD, SEQ ID NO:4 AAAFVNQHLCGSHLVEALYLVCGERGFFYTPKTR, SEQ ID NO:5 PAAAFVNQHLCGSHLVEALYLVC, SEQ ID NO:6 PAAAFVNQHLCGS, SEQ ID NO:7 CGSHLVEALYLV, SEQ ID NO:8 VEALYLVC, SEQ ID NO:9 LVCGERGF, SEQ ID NO:10 FFYTPK, SEQ ID NO:11 REAEDLQVGQVELGGGPGAGSLQPLALEGSL, SEQ ID NO:12 REAEDLQVGQVE, SEQ ID NO:13 LGGGPGAG, SEQ ID NO:14 SLQPLALEGSL, SEQ ID NO:15 GIVEQCCTSICSLYQLENYCN, SEQ ID NO:16 GIVEQCCTSICSLY, SEQ ID NO:17 QLENYCN, and SEQ ID NO:18 CSLYQLE variation within 75% exact homology. Variations include natural and modified aminoacids.

The rare molecule fragments of insulin can be used to measure the peptidases acting on insulin based on formation of fragments. This includes the list of natural known peptidase and others added to the biological system. Additional rare molecule fragments of insulin of can be used to measure inhibitors for peptidases acting on insulin based on the lack formation of fragments. These inhibitor include the c-terminal fragment of the Adiponectin Receptor, Bikunin, Uristatin and other known natural and synthetic inhibitors of archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, and matrix metallopeptidase-9 listed in the inhibitor databases.

Rare molecule fragments of bioactive therapeutic proteins and peptides can be used to measure the presence or absence thereof as an indication of therapeutic effectiveness, stability, usage, metabolism, action on biological pathways (such as actions with proteases, peptidase, enzymes, receptors or other biomolecules), action of inhibition of pathways and other interactions with biological systems. Examples include but are not limited to those listed in databases of approved therapeutic peptides and proteins, such as http://crdd.osdd.net/ as well as other databases of peptides and proteins for dietary supplements, probiotics, food safety, veterinary products, and cosmetics usage. The list of the 467 approved peptide and protein therapies include examples of bioactive proteins and peptides for use in cancer, metabolic disorders, hematological disorders, immunological disorders, genetic disorders, hormonal disorders, bone disorders, cardiac disorders, infectious disease, respiratory disorders, neurological disorders, adjunct therapy, eye disorders, and malabsorption disorder. Bioactive proteins and peptides include those used as anti-thrombins, fibrinolytic, enzymes, antineoplastic agents, hormones, fertility agents, immunosupressive agents, bone related agents, antidiabetic agents, and antibodies

Some specific examples of therapeutic proteins and peptides include glucagon, ghrelin, leptin, growth hormone, prolactin, human placental, lactogen, luteinizing hormone, follicle stimulating hormone, chorionic gonadotropin, thyroid stimulating hormone, adrenocorticotropic hormone, vasopressin, oxytocin, angiotensin, parathyroid hormone, gastrin, buserelin, antihemophilic factor, pancrelipase, insulin, insulin aspart, porcine insulin, insulin lispro, insulin isophane, insulin glulisine, insulin detemir, insulin glargine, immunglobulins, interferon, leuprolide, denileukin, asparaginase, thyrotropin, alpha-1-proteinase inhibitor, exenatide, albumin, coagulation factors, alglucosidase alfa, salmon calcitonin, vasopressin, dpidermal growth factor (EGF), cholecystokinin (CCK-8), vacines, human growth hormone and others. Some new examples of therapeutic proteins and peptides include GLP-1-GCG, GLP-1-GIP, GLP-1, GLP-1-GLP-2, and GLP-1-CCKB

Rare molecules of interest that are highly expressed by adipose tissue include but are not limited to ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Rare molecules of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adrenocorticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine betamonooxygenase), HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare molecules of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Rare molecules of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, C1orf61 chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare molecules of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyrotropin-releasing hormone

Rare molecules of interest that are highly expressed by gastrointestinal tract, salivary gland, esophagus, stomach, duodenum, small intestine, or colon include but are not limited to GKN1 Gastrokine 1, GIF Gastric intrinsic factor (vitamin B synthesis) , PGA5 Pepsinogen 5 group I (pepsinogen A), PGA3 Pepsinogen 3, group I (pepsinogen A, PGA4 Pepsinogen 4 group I (pepsinogen A), LCT Lactase, DEFAS Defensin, alpha 5 Paneth cell-specific, CCL25 Chemokine (C—C motif) ligand 25, DEFA6 Defensin alpha 6 Paneth cell-specific, GAST Gastrin, MS4A10 Membrane-spanning 4-domains subfamily A member 10, ATP4A and ATPase, H+/K+ exchanging alpha polypeptide.

Rare molecules of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Rare molecules of interest that are highly expressed by kidney include but are not limited to UMOD uromodulin, TMEM174 transmembrane protein 174, SLC22A8 solute label family 22 (organic anion transporter) member 8, SLC12A1 solute label family 12 (sodium/potassium/chloride transporter) member 1, SLC34A1 solute label family 34 (type II sodium/phosphate transporter) member 1, SLC22A12 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Rare molecules of interest that are highly expressed by lung include but are not limited to SFTPC surfactant protein C, SFTPA1 surfactant protein Al, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 protooncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare molecules of interest that are highly expressed by liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6.

Rare molecules of interest that are highly expressed by testis or prostate include but are not limited to PRM2 protamine 2 PRM1 protamine 1 TNP1 transition protein 1 (during histone to protamine replacement) TUBA3C tubulin, alpha 3c LELP1late cornified envelope-like proline-rich 1 BOD1L2 biorientation of chromosomes in cell division 1-like 2 ANKRD7 ankyrin repeat domain 7 PGK2 phosphoglycerate kinase 2 AKAP4 A kinase (PRKA) anchor protein 4 TPD52L3 tumor protein D52-like 3 UBQLN3 ubiquilin 3 and ACTL7A actin-like 7A.

### Examples of Rare Cells and Rare Cell Markers

Rare cells are those cells that are present in a sample in relatively small quantities when compared to the amount of non-rare cells in a sample. In some examples, the rare cells are present in an amount of about 10⁻⁸% to about 10⁻²% by weight of a total cell population in a sample suspected of containing the rare cells. The phrase “cellular rare molecules” refers to rare molecules that are bound in a cell and may or may not freely circulate in a sample. Such cellular rare molecule include biomolecules useful in medical diagnosis of diseases as above and also include all rare molecules and uses previously described as cell free rare molecules and those for biomolecules used for measurement of rare cells. The rare cells may be, but are not limited to, malignant cells such as malignant neoplasms or cancer cells; circulating cells, endothelial cells (CD146); epithelial cells (CD326/EpCAM); mesochymal cells (VIM), bacterial cells, virus, skin cells, sex cells, fetal cells; immune cells (leukocytes such as basophil, granulocytes (CD66b) and eosinophil, lymphocytes such as B cells (CD19,CD20), T cells (CD3,CD4 CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or other for example.

The phrase “population of cells” refers to a group of cells having an antigen or nucleic acid on their surface or inside the cell where the antigen is common to all of the cells of the group and where the antigen is specific for the group of cells. Such an antigen or nucleic acid is termed a “rare cell marker”. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells, for example.

The term “rare cell marker” includes, but is not limited to, cancer cell type biomarkers, cancer bio markers, chemo resistance biomarkers, metastatic potential biomarkers, and cell typing markers, cluster of differentiation (cluster of designation or classification determinant, often abbreviated as CD) which is a protocol used for the identification and investigation of cell surface molecules providing targets for immunophenotyping of cells. Cancer cell type biomarkers include, by way of illustration and not limitation, cytokeratins (CK) (CK1, CK2, CK3, CK4, CKS, CK6, CK7, CK8 and CK9, CK10, CK12, CK 13, CK14, CK16, CK17, CK18, CK19 and CK2), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CA1X, MIB1, MDM, PR, ER, SELS, SEMI, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDHL NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNAll, KRAS, PTPN11, DDR2, CTNNB1, GNAQ, MET, RBI, AKT1, BRAF, DDR2, MEK1, NRAS, FGFR1, and ROS1, for example.

In certain embodiments, the rare cells may be endothelial cells which are detected using markers, by way of illustration and not limitation, CD136, CD105/Endoglin, CD144/VE-cadherin, CD145, CD34, Cd41 CD136, CD34, CD90, CD31/PECAM-1, ESAM,VEGFR2/Fik-1, Tie-2, CD202b/TEK, CD56/NCAM, CD73/VAP-2, claudin 5, ZO-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARα), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7, CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

The rare molecules from cells may be from any organism, which include but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesenchymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject, for example.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11 c, CD14, CD15, CD16, CD19, CD20, CD31, CD33, CD45, CD52, CD56, CD 61, CD66b, CD123, CTLA-4, immunoglobulin, protein receptors and cytokine receptors and other CD marker that are present on white blood cells can be used to indicate that a cell is not a rare cell of interest.

In particular non-limiting examples white blood cell markers include CD45 antigen (also known as protein tyrosine phosphatase receptor type C or PTPRC) and originally called leukocyte common antigen is useful in detecting all white blood cells. Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+,CD3+, CD4+; cytotoxic T cells are indicated by CD45+,CD3+, CDS+; B-lymphocytes are indicated by CD45+, CD19+ or CD45+, CD20+; thrombocytes are indicated by CD45+, CD61+; and natural killer cells are indicated by CD16+, CD56+, and CD3−. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all markers and are for example only.

In some cases, rare molecule fragments of lymphocytes include proteins and peptides produced as part of lymphocytes such as immunoglobulin chains, major histocompatibility complex (MHC) molecules, T cell receptors, antigenic peptides, cytokines, chemokines and their receptors (e.g, Interluekins, C—X—C chemokine receptors, etc), programmed death-ligand and receptors (Fas, PDL1, and others) and other proteins and peptides that are either parts of the lymphocytes or bind to the lymphocytes.

In other cases the rare cell may be a stem cell and include but are not limited to the rare molecule fragment of stem markers cells including, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sa114, Stat3, SUZ12, TCF3, TRA-1-60, CDX2, DDX4, Miwi, Mill GCNF, Oct4, Klf4, Sox2, c-Myc, TIF 1βPiwil, nestin, integrin, notch, AML, GATA, Esrrb, Nr5a2, C/EBPα, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii. Faecalibacterium prauznitzli, Lactobacillus gasseria, Streptococcus mutans, Bacteroides thetaiotaomicron, Prevotella Intermedia, Porphyromonas gingivalis Eubacterium rectale Lactobacillus amylovorus, Bacillus subtilis, Bifidobacterium longum Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MRSA, for example; and sexually transmitted diseases. In the case of detecting rare cell pathogens, a capture particle is added that comprises an affinity agent, which binds to the rare cell pathogen population. Additionally, for each population of cellular rare molecules on the pathogen, a reagent is added that comprises an affinity agent for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

### Kits for Conducting Methods

The apparatus and reagents for conducting a method in accordance with the principles described herein may be present in a kit useful for conveniently performing the method. In one embodiment, a kit comprises in packaged combination, a modified affinity agent for one or more different rare molecules to be isolated. The kit may also comprise one or more affinity agents for cellular rare molecules, the porous matrix, capture particles, and solutions for spraying, filtering and reacting the analytical labels. The composition of the label particle may be, for example, as described above for capture particle entities. Porous matrix and electrode can be in an assembly where the assembly can have vents, capillaries, chambers, liquid inlets and outlets. The porous matrix can be remove-able or permanently fixed to the assembly.

Depending on the method used for analysis of rare molecules, reagents discussed in more detail herein below may or may not be used to treat the samples during, prior or after the extraction of molecules from the rare cells and cell free samples.

The relative amounts of the various reagents in the kits can be varied widely to provide for concentrations of the reagents that substantially optimize the reactions that need to occur during the present methods and further to optimize the sensitivity of the methods. Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The spray solvent can be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethylsulphoxide, and nitromethane; non-polar organic compounds such as, e.g., hexane, toluene, cyclohexane; and water, for example, or combinations of two or more thereof. Optionally, the solvents may contain one or more of an acid or a base as a modifier (such as, volatile salts and buffer, e.g., ammonium acetate, ammonium bicarbonate, volatile acids such as formic acid, acetic acid, trifluoroacetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the above mentioned spray solvents might be used in combination with aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is a moderate pH ranging from about 4 to about 9. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

Cell lysis reagents are those that involve disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations or two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples the amount of the lytic agent is (percentages are by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

Removal of lipids may be carried out using, by way of illustration and not limitation, detergents, surfactants, solvents, and binding agents, and combinations of two or more of the above. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, it may be desirable to remove or denature proteins from the cells, which may be accomplished using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 60%, or at least about 70%, or at least about 80%, or at least about 90%, or at least about 95% of proteins in the cells. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, samples are collected from the body of a subject into a suitable container such as, but not limited to, a cup, a bag, a bottle, capillary, or a needle, for example. Blood samples may be collected into Vacutainer® containers, for example. The container may contain a collection medium into which the sample is delivered. The collection medium may be either dry or liquid and may comprise an amount of platelet deactivation agent effective to achieve deactivation of platelets in the blood sample when mixed with the blood sample.

Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes, for example.

In many examples, the above combination is provided in an aqueous medium, which may be solely water or which may also contain organic solvents such as, for example, polar aprotic or protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 5 mL to about 100 mL, or about 5 mL to about 80 mL, or about 5 mL to about 60 mL, or about 5 mL to about 50 mL, or about 5 mL to about 30 mL, or about 5 mL to about 20 mL, or about 5 mL to about 10 mL, or about 10 mL to about 100 mL, or about 10 mL to about 80 mL, or about 10 mL to about 60 mL, or about 10 mL to about 50 mL, or about 10 mL to about 30 mL, or about 10 mL to about 20 mL, or about 20 mL to about 100 mL, or about 20 mL to about 80 mL, or about 20 mL to about 60 mL, or about 20 mL to about 50 mL, or about 20 mL to about 30 mL, for example.

Where one or more of the rare molecules are part of a cell, the aqueous medium may also comprise a lysing agent for lysing of cells. A lysing agent is a compound or mixture of compounds that disrupt the integrity of the matrices of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the proteins, peptides, nucleic acids or cells of the sample. Fixation immobilizes and preserves the structure of proteins, peptides and nucleic acids and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells, for example. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde,); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both, for example.

In some examples utilizing the techniques, it may be necessary to subject the rare cells to permeabilization. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells, for example. In some examples, the amount of permeabilization agent by weight is about 0.1% to about 0.5%, or about 0.1% to about 0.4%, or about 0.1% to about 0.3%, or about 0.1% to about 0.2%, or about 0.2% to about 0.5%, or about 0.2% to about 0.4%, or about 0.2% to about 0.3%, for example. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20); for example. One or more washing steps may be carried out on the permeabilized cells using a buffered aqueous medium.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted.

### Abbreviations:

WBC=white blood cells
DAPI=4′,6-diamidino-2-phenylindole
DMSO=dimethylsulfoxide (ThermoFisher Scientific)
min=minute(s)
μm=micron(s)
mL=milliliter(s)
mg=milligrams(s)
μg=microgram(s)
PBS=phosphate buffered saline (3.2 mM Na₂HPO₄, 0.5 mM KH₂PO₄, 1.3 mM KCl, 135 mM NaCl, pH 7.4)
K₃EDTA=potassium salt of ethylenediaminetetraacetate
mBar=millibar
w/w=weight to weight
RT=room temperature
hr=hour(s)
QS=quantity sufficient
ACN=acetonitrile
TFA=trifluoroacetic acid
TCEP=tris(2-carboxyethyl)phosphine hydrochloride (Sigma-Aldrich)
SPDP=N-Succinimidyl 3-(2-pyridyldithio)propionate)
SH-NeutrAvidin=sulfhydryl-modified neutravidin
NeutrAvidin=affinity agent for biotin
Ab=antibody
mAb=monoclonal antibody
vol=volume
MW=molecular weight
wt.=weight
Analyte cells=SKBR3 human breast cancer cells (ATCC)
Her2nue=Human epidermal growth factor receptor 2
Variations of analyte=Her2nue obtained from lyzed SKBR3 human breast cancer cells (ATCC)
Affinity agent for Her2nue=Monoclonal anti Her2nue antibody (NB3 clone) (ATCC)
Label particle=Propylamine-functionalized silica nano-particles 80 nm,
Glass slide=FISHERBRAND™ SUPERFROST™ Plus Microscope Slides (ThermoFisher Scientific Inc.)
Blocking agent=Casien, the blocking solution (Candor Biosience GmbH, Allgau Germany)
Capture particles=BioMag® hydroxyl silica micro particles (46.2 mg/mL, 1.5 μm) with streptavidin (Bangs Lab Inc.) with anti Her2nue antibody (NB3 clone from ATCC) made by direct conjugation to the particles.
Magnet=Dynal magnetic particle concentrator
Porous Matrix=WHATMAN® NUCLEOPORE™ Track Etch matrix, 25 mm diameter and 8.0 and 1.0 μM pore sizes
MS=Mass spectroscopy analysis by nano electrospray ionization on a Thermo LTQ (linear ion trap) mass spectrometer (from Thermo Electron North America LLC).

The following examples are in accordance with the principles described herein, where methods of isolation of variations of analyte molecules in a sample by binding variations to a particle through an affinity agent attached to particle by an X-Y bond which is also attached to analytical labels by an X-Y bond and separating the particles from the sample followed by removing analytical labels from particle and measuring the analyte molecules by the measuring analytical labels after releasing by conditions breaking the X-Y bond to the analytical label.

### Example 1

### Particle Attachment of Analytical Labels and Affinity Agent by an X-Y Bond

Attachment of affinity agents and analytical labels by an X-Y bond is shown in the following example which utilizes an —S—S— bond (disulfide). In this example, aminated silica nanoparticles (label particles) were suspended in DMSO to a final concentration of 20 mg/mL. SPDP was dissolved in DMSO in a separate tube to a final concentration of 20 nmole/μL. The SPDP stock solution was added dropwise to the 20 mg/mL aminated silica nanoparticles in DMSO while gently swirling. The mixture was allowed to react for at least 60 minutes at RT with constant mixing. Following the reaction time, the reaction mix was centrifuged, the supernatant removed and discarded and the particles were resuspended in DMSO. This washing procedure was repeated 3 additional times following which the SPDP reacted nanoparticles were resuspended by sonication to a final concentration of 3.3 mg/mL.

Peptide comprising a free SH (analytical label) was dissolved in PBS-EDTA. NeutrAvidin (affinity tag), previously modified to contain an average of one free thiol (via conjugation with Traut's reagent) per NeutrAvidin was added to the solution containing the analytical label. The final concentration of analytical label and NeutrAvidin was approximately 1 mM and 20 respectively. To the solution of SH-peptide/SH-neutravidin was added the suspension of SPDP modified nanoparticles in DMSO and the reaction was allowed to incubate at room temperature with stirring overnight. After the reaction, the particles were washed three time with PBS and resuspended into 1 mL PBS.

In order to make X-Y bonds for cases when the X are metals such as Ni, Co, Fe or Cu, the silica amine nanoparticle could be conjugated to chelating agent like ethylenediaminetetraacetic acid (EDTA) or others, to allow binding of the metal. In order to make X-Y cases when the X are metals such as Pd, Ag, or Au, the silica amine nanoparticle is conjugated to sulfhydryl (—SH) groups as a chelating agent to allow binding of the metal. The metals conjugated to the silica amine label were attached to affinity agent, or analytical label using through a Y which is a S, O, C, P, N, B, Si by the formation of bonds which are sulfides, ethers, esters, thioesters, amides, ketals, thioamides, N-oxides, nitrogen-nitrogen, or thioethers. These bonds were formable by standard chelate metalorganic chemistry such as O, C, P, N, or B anion to form a bond to the metal group In order to make X-Y bonds for cases when the X are organic atom such as O, C, P, N, or B, the silica amine nanoparticle was conjugated to linkage agent where the X group was attached to a carboxylic acid and Y group is attached to an amine group. The carboxylic acid was attached to the silica amine nanoparticle and the amine group was attached to the affinity agent and analytical labels. The X-Y bond could then be varied to include, —S—S— sulfides, —C—O— ethers, —[C═O]—O—C— esters, —[C═O]—S—C— thioesters, —[C═O]—N—C amides, (—C—O—)₂ ketals, [C═O]—N—S thioamides, —N—O—N-oxide, —N—N— nitrogen-nitrogen, or —S—O— thioethers.

### Example 2

### Isolation of Variations of Analyte Molecules with Particle from Example 1

Isolation of variations of analyte molecules by binding variations to a particle through an affinity agent is shown in the following example which uses human epidermal growth factor receptor 2 (Her2nue) as an example of variations of analyte molecules in a sample. The Her2nue proteins was found to be cleaved and converted to many variations by the mechanism shown in FIG. 1. The isolation is demonstrated by binding variations to the particles through an affinity agent for Her2nue which was conjugated to biotin (affinity tag).

In this example, NeutrAvidin served as an affinity agent for biotin and is bound to the particle by an X-Y bond, in this example an S—S— bond. The label particles also have attached analytical labels by the same S—S bond. In this example the separation of particles from the sample are demonstrated in two means. In a first case, the particle is bound to Her2nue variations on a cell, namely SKBR3 cells, and the bound particles are separated with the cell via size-exclusion filtration. In a second case, the particle is bound to Her2nue binding variations that are free of cells, namely from lysed SKBR cells, and the Her2nue bound to particles are separated with a capture particle with a second affinity agent for Her2nue. The capture particles are removed with the Her2nue bound to particles by magnetic forces.

The Her2nue proteins were prepared in a cellular form by centrifuging 500 μL of a solution containing approximately 2×105 cancer cells (SKBR3) cells/mL. About 1 mL of PBS was added to wash the cell pellet by inverting the tube several times to mix, centrifuging again at relative centrifugal forces of 2000 for 3 min and removing wash liquid. The cells were permeabilized by adding 1 mL of 0.2% Triton-X in PBS, the tube inverted several times and incubated for 7 minutes followed by washing. The cells were blocked to reduced non-specific binding by adding 1 mL of fragmented casein buffer and the mixture vortexed gently to mix. The mixture was centrifuged again, the liquid removed and the was step repeated once more. The cell mixture was diluted to 1 mL with PBS and a 10 μL sample was examined under the microscope to determine a cell count. The Her2nue proteins were prepared in a cell free form by lysing the cells. The samples for testing were prepared by collecting blood from healthy donors (9 mL per donor) and stored in Transfix tubes for up to 5 days. The blood sample was spiked with Her2nue variations which were SKBR3 human breast cancer cells (ATCC) cell using a stock to give 1000 cells/0.5 mL. A second blood sample was also spiked with about ˜1000 lysed SKBR3 cells into 0.5 mL blood to provide cell free variations of analyte molecules.

For isolation of variations of cell free Her2nue molecules, the sample with lysed SKBR3 cells was first captured on capture particles (magnetic beads conjugated with anti Her2nue antibody) by adding 50 μL of capture particles to the 1 mL sample. Samples were mixed by inverting, and the mixture incubated at RT for 15 minutes to allow the particles to capture the variations of cell free Her2nue molecules. This was followed by addition of label particle along with an additional Her2neu affinity agent. Capture particles were isolated by centrifuging the tube at 1700 g for 3 minutes (or filtration on a porous membrane with 1 μm pore or captured to the wall of vial with a magnet) and the supernatant removed. Magnetic beads were diluted with 250 μL PBS to suspend the pellet of beads. The particles were washed 5 times with PBS.

For isolation of variations of cellular Her2nue molecules, the SKBR3 cells were first captured on a porous matrix using a vacuum to provide a hydrodynamic force according to previous published methods (Pugia el al, A Novel Strategy for Detection and Enumeration of Circulating Analyte Cell Populations in Metastatic Cancer Patients Using Automated Fluidic Filtration and Multiplex Immunoassay PLoS ONE 014166 (2015)). The whole blood with intact SKBR3 cells and WBC were diluted in PBS, and filtered through according to the filtration process as previously described. The only change to the process was to use a vacuum filtration unit (Biotek Inc) for a standard ELISA plate fitted with the porous matrix. The sample was filtered through a membrane with 8.0 μm pores. During filtration, sample on the porous matrix was subjected to a negative pressure, that is, a decrease greater than about −100 mBar from atmospheric pressure. The vacuum applied varied from −10 to −100 mBar during filtration. The diluted sample was placed into the filtration station without mixing and the diluted sample was filtered through the porous matrix. Recovery of SKBR3 cells were >60% for each sample.

The isolated SKBR3 cells were then reacted with label particle by affinity reaction is performed and according to previous published methods and particles. In summary, following the filtration, the porous matrix was washed with PBS, and the sample was fixed with formaldehyde, washed with PBS, subjected to permeabilization using of 0.2% TRITON® X100 in PBS and washed again with PBS. A blocking step was employed in which blocking buffer of 10% casein in PBS was dispensed on the matrix. After an incubation period of 5 min, the matrix was washed with PBS to block non-specific binding to the matrix. The blocking step and permeabilization step were performed for the first affinity reaction and not repeated for second and third affinity reactions. Five PBS TWEEN® surfactant washings were done after each affinity reaction. The rare cells were then measured using affinity reactions and immunocytochemistry (ICC) with a flourecent label attached to the antibody for CK8/18. The mAb to Her2nue was bound to SBKR cell and not the WBC as demonstrated by the microscope showing the presence of Dy550 only in SBKR3 cells.

In both cases, samples were contaminated with non-rare molecule, such a white blood (WBC) and red blood cells (RBC). In the cell case, the purity of the SBKR cells in WBC was between 0.1 and 0.01%. A high percentage Her2nue molecules variations (>80%) were captured whether using the capture particles or in the SBKR3 cells with antibodies binding to fragments of interest and not to contaminating WBC or RBC. The method worked whether more Her2nue affinity agents (TA1 or NB3 clones) were attached to the same particle and when with different affinity agents and unique analytical labels are attached to different particles.

### Example 3

### Removal of Analytical Labels by Breaking X-Y Bond from Particle

Isolated cells or particles were first treated with a reagent to break the X-Y bond and release the analytical label from the label particle. In the case of an X-Y bond of —S—S—, the sample was treated with 10 μL of a release solution (10 mM TCEP, 5 nM internal standard in 10 mM ammonium acetate buffer, pH 4.5) to release the analytical label. Analysis by mass spectroscopy (MS) demonstrated >90% capture and release efficiencies. A series of experiments was performed to calculate analytical sensitivity to detect cell and cell free Her2nue molecules variations in a whole blood sample. The observed analytical sensitivity was determined by measurements of samples with 0, 50, 100, 200, 500, and 1000 intact or lysed SKBR3 cells added to whole blood. The methods limit of was determined as 10 times the signal of the zero level and by confirmation by optically counting the number of cell capture by microscopic technique. Multiple types of analytical labels, microscopic optical, mass spectroscopic, chemiluminescent, electrochemical and microscopic fluorescence read out were used and limits of detection were comparable and the typical limit of detection is reported in Table 1. Additionally the cell and cell free limits of detection were comparable and the typical limit of detection is reported in Table 1.

TABLE 1
Comparison of limits of detections

| | —X—Y— | | —X—Y | Limit |
|---|---|---|---|---|
| | bond to | —X—Y bond to | bond to | of detection |
| Case | label | affinity agent | affinity tag | (cells) |
| 1 | Non- | Non-breakable | None | ~5000-10,000 |
| | breakable | | | |
| 2 | Breakable | Breakable | None | ~100-400 |
| 3 | Breakable | None | Breakable | ~1000-3000 |
| 4 | Breakable | Non-breakable | None | ~100-400 |
| 5 | Breakable | Breakable (multiple | None | ~10-100 |
| | | antibodies) | | |
| 6 | Breakable | Breakable (multiple | None | ~10-100 |
| | | particles) | | |

The limits of detection are shown in the data in Table 1 for examples 2, 4, 5 and 6 which are in accordance with the principles described herein and are directed to methods of isolation of variation of analyte in a sample by binding all variation of analyte to particle with analytical label; where multiple identical affinity agents are attached to particle by and X-Y bond but are not released by conditions breaking the X-Y bond. In examples 1, the use of non-breakable X-Y bond, the method was much less sensitive and is unable to detect the ˜100-400 of SKBR3 cells in comparison to example 2 in which the X-Y bonds are breakable. Surprisingly, if the affinity agent on the particle is replaced with an affinity tag, as in example 3, the method is unable to detect the ˜100-400 of example 2. As expected if multiple affinity agents are used on the particle, as in example 5, or multiple particles with different affinity agents are used, as in accordance with the invention, the method is able to detect even less cells than the ˜100-400 of example 2. Additionally if the X-Y bond to the affinity agent does not break, the number of cells detected remains the same as example 2. Overall this demonstrates the benefits of the invention to have analytical labels and affinity agents are attached to particle by and X-Y bond.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

### REFERENCES

1. Karen A. Sap and Jeroen A. A. Demmers (2012). Labeling Methods in Mass Spectrometry Based Quantitative Proteomics, Integrative Proteomics, Dr. Hon-Chiu Leung (Ed.), ISBN: 978-953-51-0070-6, InTech, Available from: http://www.intechopen.com/books/integrative-proteomics/labeling-methods-in-mass-spectrometry-basedquantitative-proteomics
2. Y. Zhu, R. Valdes Jr., C. Q. Simmons, M. W. Linder, M. J. Pugia, S. A. Jortani. Analysis of Ligand binding by Bioaffinity Mass Spectrometry. Clin Chem Acta 371(1-2), 71-8 (2006).
3. Robert Popp⋅David Malmstrom⋅Andrew G Chambers, D. Lin, A. G Camenzind, J Grace van der Gugten, D. T. Holmes, M. Pugia, M. Jaremek, S Cornett , D. Suckau, C H Borchers AAn Automated Assay for the Clinical Measurement of Plasma Renin Activity by immuno-MALDI (iMALDI). Biochimica et Biophysica Acta—Proteins & Proteomics 10/; 1854(6) (2014).
4. Dmitry R. Bandura, Vladimir I. Baranov, Olga I. Ornatsky, Alexei Antonov, Robert Kinach, Xudong Lou, Serguei Pavlov, Sergey Vorobiev, John E. Dick, and Scott D. Tanner. Mass Cytometry: Technique for Real Time Single Cell Multitarget Immunoassay Based on Inductively Coupled Plasma Time-of-Flight Mass Spectrometry. Anal. Chem. 2009, 81, 6813-6822
5. Jung Rok Lee, Juhee Lee, Sang Kyung Kim, Kwang Pyo Kim, Hyung Soon Park, Woon-Seok Yeo. Mass Spectrometry Signal Amplification Method for Attomolar Detection of Antigens Using Small-Molecule-Tagged Gold Microparticles Angew. Chem. Int. Ed. 2008, 47, 9518-9521
6. M. Pugia et all Immunological evaluation of urinary trypsin inhibitors in blood and urine: Role of N- & O-linked glycoproteins Glycoconj J (2007) 24:5-15
7. Commonly owned pending U.S. application Ser. No. 15/941,059 entitled Methods And Apparatus For Removal Of Small Volume From A Filtration Device filed Mar. 30, 2018 and Ser. No. 15/941,125 entitled Methods And Apparatus For Selective Nucleic Acid Analysis filed Mar. 30, 2018 all incorporated by reference herein.

What is claimed is:

1. A method of isolating and measuring variations in an analyte sample, said method comprising:
(a) binding said analyte sample having variations to a particle having attached analytical labels;
(b) separating the resulting particles from the sample;
(c) removing the analytical labels from the particle; and
(d) measuring the analyte molecules by the measuring analytical labels.

2. The method of claim 1, wherein the analytical labels are attached to the particle by and X-Y bond and released by breaking the X-Y bond.

3. The method of claim 1, wherein variations of said analyte are bound to said particle by one or more affinity agents.

4. The method of claim 1, wherein said affinity agents are attached by an X-Y bond and released by breaking the X-Y bond.

5. The method of claim 2, wherein the X-Y bond used to attach the analytical label are sulfides, ethers, esters, thioesters, amides, ketals, thioamides, N-oxides, nitrogen-nitrogen, or thioethers.

6. The method of claim 4, wherein the X-Y bond used to attach the affinity agent are sulfides, ethers, esters, thioesters, amides, ketals, thioamides, N-oxides, nitrogen-nitrogen, or thioethers.

7. The method of claim 2, wherein X-Y are selected from the group consisting of S, O, C, P, N, B, Si, Ni, Pd, Fe Co, Ag, Cu, or Au.

8. The method of claim 4, wherein X-Y are selected from the group consisting of S, O, C, P, N, B, Si, Ni, Pd, Fe Co, Ag, Cu, or Au.

9. The method of claim 2, wherein the X-Y bond can be part of a long linker group to cause space between the affinity agent, or analytical label and the label particle.

10. The method of claim 3, wherein said affinity agents to multiple variations of analyte are attached to the same particle.

11. The method of claim 1, wherein multiple particles bind variations with different affinity agents and having analytical labels attached to the particle.

12. The method of claim 1, wherein variation of the analyte can be man-made or of natural origin.

13. The method of claim 1, wherein variation of the analyte can be bioactive, or non-bioactive molecules.

14. The method of claim 1, wherein variation of the analyte can be cellular or free of cells.

15. The method of claim 1, wherein variation of the analyte can be measurements of other molecules causing inhibition variation.

16. The method of claim 1, wherein variation of analyte can be intentional or generated by fragmentation, addition or binding.

17. The method of claim 1, wherein variation of said analyte can be a metabolite, co-factors, substrates, amino acids, metals, vitamins, fatty acids, biomolecules, peptides , carbohydrate or others as well as macromolecules, like glycoconjugates, lipid, nucleic acids, polypeptides, receptors, enzymes, protein as well as cells and tissues including cellular structures, peroxisomes, endoplasmic reticulum, endosomes, exosomes, lysosomes, mitochondria, cytoskeleton, membranes, nucleus, extra cellular matrix or other molecule typically measured.

18. The method of claim 1, wherein particles binding variation of analyte are removed by a porous matrix, a capture particle, a cell or magnetic particle or combinations thereof.

19. The method of claim 1, wherein analytical labels are detected by mass spectroscopy, fluorescence, chemiluminescence or optically labels or combinations thereof.


SEQUENCE LISTING (25 sequences)

SEQ ID NO: 1 - protein, 19 aa, Artificial Sequence
  Synthetic
MALWMRLLPLLALLALWGP

SEQ ID NO: 2 - protein, 10 aa, Artificial Sequence
  Synthetic
MALWMRLLPL

SEQ ID NO: 3 - protein, 9 aa, Artificial Sequence
  Synthetic
ALLALWGPD

SEQ ID NO: 4 - protein, 34 aa, Artificial Sequence
  Synthetic
AAAFVNQHLCGSHLVEALYLVCGERGFFYTPKTR

SEQ ID NO: 5 - protein, 23 aa, Artificial Sequence
  Synthetic
PAAAFVNQHLCGSHLVEALYLVC

SEQ ID NO: 6 - protein, 13 aa, Artificial Sequence
  Synthetic
PAAAFVNQHLCGS

SEQ ID NO: 7 - protein, 12 aa, Artificial Sequence
  Synthetic
CGSHLVEALYLV

SEQ ID NO: 8 - protein, 8 aa, Artificial Sequence
  Synthetic
VEALYLVC

SEQ ID NO: 9 - protein, 8 aa, Artificial Sequence
  Synthetic
LVCGERGF

SEQ ID NO: 10 - protein, 6 aa, Artificial Sequence
  Synthetic
FFYTPK

SEQ ID NO: 11 - protein, 31 aa, Artificial Sequence
  Synthetic
REAEDLQVGQVELGGGPGAGSLQPLALEGSL

SEQ ID NO: 12 - protein, 12 aa, Artificial Sequence
  Synthetic
REAEDLQVGQVE

SEQ ID NO: 13 - protein, 8 aa, Artificial Sequence
  Synthetic
LGGGPGAG

SEQ ID NO: 14 - protein, 11 aa, Artificial Sequence
  Synthetic
SLQPLALEGSL

SEQ ID NO: 15 - protein, 21 aa, Artificial Sequence
  Synthetic
GIVEQCCTSICSLYQLENYCN

SEQ ID NO: 16 - protein, 14 aa, Artificial Sequence
  Synthetic
GIVEQCCTSICSLY

SEQ ID NO: 17 - protein, 7 aa, Artificial Sequence
  Synthetic
QLENYCN

SEQ ID NO: 18 - protein, 7 aa, Artificial Sequence
  Synthetic
CSLYQLE

SEQ ID NO: 19 - protein, 8 aa, Artificial Sequence
  Synthetic
WSHPQFEK

SEQ ID NO: 20 - protein, 38 aa, Artificial Sequence
  Synthetic
MDEKTTGWRGGHVVEGLAGELEQLRARLEHHPQGQREP

SEQ ID NO: 21 - protein, 20 aa, Artificial Sequence
  Synthetic
GVMPREETDSKTASPWKSAR

SEQ ID NO: 22 - protein, 8 aa, Artificial Sequence
  Synthetic
DYKDDDDK

SEQ ID NO: 23 - protein, 9 aa, Artificial Sequence
  Synthetic
YPYDVPDYA

SEQ ID NO: 24 - protein, 10 aa, Artificial Sequence
  Synthetic
EQKLISEEDL

SEQ ID NO: 25 - protein, 15 aa, Artificial Sequence
  Synthetic
KETAAAKFERQHMDE